(19) United States
(12) Patent Application Publication
(10) Pub. No.: US 2014/0011694 A1
(43) Pub. Date: Jan. 9, 2014

(54) METHODS, SYSTEMS, DATABASES, KITS AND ARRAYS FOR SCREENING FOR AND PREDICTING THE RISK OF AN IDENTIFYING THE PRESENCE OF TUMORS AND CANCERS

(71) Applicant: Olivier Couronne, New York, NY (US)

(72) Inventor: Olivier Couronne, New York, NY (US)

(73) Assignee: VIA GENOMES, INC., Wilmington, DE (US)

(21) Appl. No.: 13/977,899

(22) PCT Filed: Jan. 11, 2012
(86) PCT No.: PCT/US2012/020921

§ 371 Date: Sep. 24, 2013

Related U.S. Application Data
(60) Provisional application No. 61/431,741, filed on Jan. 11, 2011.

(51) Int. Cl. C12Q 1/68

(52) U.S. Cl. CPC C12Q 1/6886
USPC 506/9; 435/6.11; 536/24.31; 506/16

(57) ABSTRACT

The invention relates to predicting or determining risk of a tumor or cancer, or the presence or absence of a tumor or cancer, in a subject. The invention also relates to methods of correlating somatic chromosomal sequence rearrangements, such as rearrangements in synteny block sequences, with the presence or probability of a tumor or cancer. The invention further relates to monitoring progression or regression of a tumor or cancer in a subject. The invention moreover relates to organizational constructs (e.g., databases) and methods of producing organizational constructs (e.g., databases) in which a plurality of somatic chromosomal sequence rearrangements predictive of the presence of a tumor or cancer are recorded or stored, for example, to correlate the somatic chromosomal sequence rearrangements with a query sample from a sample of a subject analyzed for the presence or absence of a tumor or cancer.

### RELATED APPLICATIONS

This application claims the benefit of priority of application Ser. No. 61/431,741, filed Jan. 11, 2011, which is expressly incorporated herein by reference in its entirety.

### TECHNICAL FIELD

The invention relates to predicting or determining presence or absence of a tumor or cancer in a subject. The invention also relates to monitoring progression or regression of a tumor or cancer in a subject. The invention further relates to methods of correlating somatic chromosomal sequence rearrangements with the presence or probability of a tumor or cancer. The invention moreover relates to organizational constructs (e.g., databases) and methods of producing organizational constructs (e.g., databases) in which a plurality of somatic chromosomal sequence rearrangements predictive of the presence of a tumor or cancer are recorded or stored, for example, to correlate the somatic chromosomal sequence rearrangements with a query sample from a sample of a subject analyzed for the presence or absence of a tumor or cancer. The invention additionally relates to kits, arrays and systems for identifying samples having somatic chromosomal sequence rearrangements predictive of the presence of a tumor or cancer.

### INTRODUCTION

Many diseases, such as various cancers, disease associated with chromosomal imbalance (e.g. Patau syndrome, Down's syndrome, etc.), and certain immunological and neurological diseases are caused by genomic alterations, including point mutations, deletions, inversions, duplications, multiplications, chromosomal translocations and other rearrangements. These alterations either directly cause disease, or predispose the individuals to disease. In addition, the presence of certain alterations can determine the outcome of certain diseases. Thus, screening for the status of these alterations provides valuable information useful for diagnosis, for prognosis, and for clinical management, including elimination of unnecessary surgeries or other treatments, and improved quality of life of cancer patients. Additionally, study of these alterations may be useful in building disease-mutation correlations for drug discovery.

To illustrate, various chromosomal abnormalities have been described in prostate cancer. Among the most common reported are trisomy and hyperdiploidy (Cui et al., Cancer Genet Cytogenet 107: 51, 1998), gains of 6p, 7q, 8q, 9q, 16q (van Dekken et al., Lab Invest. 83: 789, 2003; Steiner et al., Eur Urol. 41: 167, 2002; Verhagen et al., Int J Cancer 102: 142, 2002; Brothman AJMG 115: 150, 2002), deletions of 3q, 6q, 8p, 10q, 13q, 16q, 17p, 20q (van Dekken, supra; Matsuyama et al., Aktuel Urol. 34: 247, 2003; Matsuyama et al., Prostate 54: 103, 2003; Bergerheim et al., Genes Chromosomes Cancer 3: 215, 1991), and aneusomy of chromosomes 7 and 17 (Cui, supra). Loss of heterozygosities (LOHs) at 13q14 and 13q21 were reported to be more common in tumors associated with local symptoms (Dong et al., Prostate 49: 166, 2001). Loss at 16q in combination with loss at 8p22 has been associated with metastatic prostate cancer (Matsuyama et al., Aktuel Urol. 34: 247, 2003). Several groups have reported that the number of genetic abnormalities seen correlates with worse prognosis (Brothman, Cancer Res. 50(12): 3795-803, 1990). Although trends from these studies have emerged, chromosomal findings have varied substantially from series to series, and their clinical relevancy in terms of diagnosis, prognosis and treatment are uncertain. Therefore, the clinical relevance, if any, of these genomic changes is not fully understood.

Thus, there is a need for methods for the diagnosis and/or prognosis of tumors and cancers associated with genomic alterations. Such diagnosis/prognosis methods can be used to screen and identify patients at increased risk for or have tumors or cancers and that require definitive therapy, while sparing patients with none or low grade disease from costly but unnecessary surgeries or other treatments.

### SUMMARY

The invention is based, at least in part, on the discovery that analysis of samples from tumors and cancers revealed the presence of somatic chromosomal sequence rearrangements in synteny block sequences that are not found in normal germline chromosomal sequences. These structural alterations of genomic synteny block sequences are markers for and can be correlated with an increased risk of or the presence or development of certain tumors and cancers. Accordingly, detecting the presence of somatic chromosomal sequence rearrangements in a sample allows for diagnosis, prognosis, monitoring and/or regression, progression or worsening of a tumor or cancer, (e.g., reduction or advancement to different stages, e.g., metastatic versus non-metastatic tumor or cancer), or an increased risk or predisposition towards developing a tumor or cancer, in the subject from which the sample is obtained.

In accordance with the invention, there are provided methods for predicting the presence or absence of a tumor or cancer in a subject or determining the risk of a tumor or cancer in a subject. In one embodiment, a method includes analyzing genomic nucleic acid for the presence or absence of a somatic chromosomal sequence rearrangement predictive of the presence of tumor or cancer or an increased risk of tumor or cancer (e.g., a chromosomal sequence rearrangement in a genomic synteny block sequence). The presence of the somatic chromosomal sequence rearrangement is predictive of the presence of tumor or cancer in the subject or an increased risk of tumor or cancer in the subject, whereas the absence of the somatic chromosomal sequence rearrangement is predictive of the absence of tumor or cancer in the subject or a reduced risk of tumor or cancer in the subject. In particular aspects, all or a portion of the genomic synteny block sequence is structurally rearranged to be in an altered proximity to a gene coding sequence, such as a gene coding for a protein that promotes or induces cell growth, proliferation, angiogenesis or survival, or a protein that reduces or inhibits cell death (apoptosis), growth inhibition, or survival, as such genes predispose or contribute to development or progression (e.g., metastases) of a tumor or cancer.

In accordance with the invention, there are also provided methods for monitoring progression or regression of a tumor or cancer in a subject. In one embodiment, a method includes analyzing genomic nucleic acid of a sample from a subject to determine an amount of nucleic acid comprising a somatic chromosomal sequence rearrangement indicative of a tumor or cancer (e.g., a chromosomal sequence rearrangement in a genomic synteny block sequence), and comparing the amount to an amount of nucleic acid comprising a somatic chromosomal sequence rearrangement (e.g., a chromosomal sequence rearrangement in a genomic synteny block sequence) indicative of a tumor or cancer of a prior sample. An increasing amount of the somatic chromosomal sequence rearrangement in the sample compared to the prior sample indicates progression of the tumor or cancer in the subject, whereas a decreasing amount of the somatic chromosomal sequence rearrangement in the sample compared to the prior sample indicates regression of the tumor or cancer in the subject.

In accordance with the invention, there are additionally provided methods for identifying somatic chromosomal sequence rearrangements correlating with the presence of a tumor or cancer, or with an increased risk of tumor or cancer. In one embodiment, a method includes analyzing genomic nucleic acid of a sample from a tumor or cancer to determine the presence or absence of a somatic chromosomal sequence rearrangement, comparing the a somatic chromosomal sequence rearrangement, if present, to a corresponding germline sequence, and repeating the foregoing steps for one or more additional samples from a tumor or cancer. Identification of a somatic chromosomal sequence rearrangement that is recurrent (e.g., a recurrent rearrangement such as a translocation) in multiple tumor or cancer cell genomic nucleic acid that is absent from a corresponding germline sequence identifies the somatic chromosomal sequence rearrangement as predictive of the presence of tumor or cancer or an increased risk of tumor or cancer.

In accordance with the invention, there are further provided computer-implemented methods for identifying somatic chromosomal sequence rearrangements correlating with the presence of a tumor or cancer, or with an increased risk of tumor or cancer, the methods implemented in a computer system comprising electronic storage and/or one or more processors. In one embodiment, a method includes receiving analysis of individual samples of tumor or cancer cell genomic nucleic acid, wherein the received analysis for a given sample indicates the presence or absence in the given sample of a somatic chromosomal sequence rearrangement; storing the received analysis to the electronic storage in an organizational construct in which information related to individual samples is stored in corresponding records such that the record corresponding to the given sample includes the analysis of the given sample; and processing the stored records to identify a common set of somatic chromosomal sequence rearrangements correlating with the presence of a tumor or cancer and/or with an increased risk of tumor or cancer.

In accordance with the invention, there are moreover provided systems configured to correlate somatic chromosomal sequence rearrangements with the presence of a tumor or cancer, or with an increased risk of tumor or cancer. In one embodiment a system includes electronic storage that stores analysis of individual samples of tumor or cancer cell genomic nucleic acid, wherein the stored analysis for a given sample indicates the presence or absence in the given sample of a somatic chromosomal sequence rearrangement, the stored analysis being organized in an organizational construct in which the analysis related to individual samples is stored in records corresponding to the individual samples such that the record corresponding to the given sample includes the analysis of the given sample; and one or more processors configured to identify a correlation between a common set of somatic chromosomal sequence rearrangements with the presence of a tumor or cancer and/or with an increased risk of tumor or cancer.

In accordance with the invention, there are still further provided methods of producing databases and organizational constructs that include a plurality of somatic chromosomal sequence rearrangements predictive of the presence of tumor or cancer or an increased risk of tumor or cancer. In one embodiment, a method includes analyzing tumor or cancer cell genomic nucleic acid for the presence or absence of a somatic chromosomal sequence rearrangement; and comparing the sequence arrangement to a corresponding germline sequence. The presence of the somatic chromosomal sequence rearrangement in the tumor or cancer cell genomic nucleic acid absent from a corresponding germline sequence indicates the somatic chromosomal sequence rearrangement as predictive of the presence of tumor or cancer or an increased risk of tumor or cancer, which can then be recorded or stored. The foregoing steps are optionally repeated for one or more additional somatic chromosomal sequence rearrangements, thereby producing a database or organizational construct comprising a plurality of somatic chromosomal sequence rearrangements predictive of the presence of tumor or cancer or an increased risk of tumor or cancer.

In accordance with the invention, there are yet additionally provided systems configured to identify samples having somatic chromosomal sequence rearrangements indicative of the presence or increased risk of a tumor or cancer. In one embodiment, a system includes electronic storage storing a plurality of somatic chromosomal sequence rearrangements indicative of a tumor or cancer; and one or more processors configured to receive analysis of a sample indicating the presence or absence one or more somatic chromosomal sequence rearrangements in the sample, to compare any somatic chromosomal sequence rearrangements in the sample with the stored plurality of somatic chromosomal sequence rearrangements indicative of a tumor or cancer, and, responsive to a somatic chromosomal sequence rearrangements in the sample matching one of the stored somatic chromosomal sequence rearrangements, to identify the sample as having a tumor or cancer.

As set forth herein, sequence rearrangements can be in somatic chromosomal sequences. Exemplary sequence rearrangements are intra-chromosomal or inter-chromosomal rearrangements. Non-limiting examples of sequence rearrangements are sequence translocations, tandem or non-tandem duplications, inverted duplications, or deletions.

Exemplary sequence rearrangements occur in genomic synteny block sequences, which are typically conserved chromosomal sequences, for example, between different species (e.g., vertebrates, such as a human, mouse and/or chicken). Genomic synteny block sequences typically include conserved non-coding and/or coding sequences, segments and elements.

In more particular aspects, a sequence rearrangement occurs in any of: chromosome 1, in a sequence region from about 79,177,716 to about 84,414,777; chromosome 1, in a sequence region from about 56,498,495 to about 59,005,059; chromosome 2, in a sequence region from about 5,174,608 to about 9,099,558; chromosome 2, in a sequence region from about 57,825,183 to about 61,899,453; chromosome 3, in a sequence region from about 72,517,657 to about 74,474,129; chromosome 5, in a sequence region from about 156,565,132 to about 158,632,403; chromosome 6, in a sequence region from about 7,047,303 to about 9,164,260; chromosome 7, in a sequence region from about 155,264,117 to about 157,210,205; chromosome 8, in a sequence region from about 92,587,940 to about 94,938,420; chromosome 11, in a sequence region from about 30,351,542 to about 32,975,808; chromosome 12, in a sequence region from about 41,040,453 to about 45,974,198; chromosome 13, in a sequence region from about 53,236,066 to about 55,250,543; chromosome 13, in a sequence region from about 58,902,901 to about 61,141,887; chromosome 15, in a sequence region from about 94,878,945 to about 99,073,175; chromosome 16, in a sequence region from about 6,703,581 to about 9,024,395; chromosome 18, in a sequence region from about 18,877,624 to about 23,308,408; chromosome 19, in a sequence region from about 30,115,800 to about 33,770,238, of all or a part of any of the foregoing genomic synteny block sequences. The wherein numerical coordinates for genomic synteny block sequence are as defined in the Human Genome Reference Consortium, Version GRCh37.

Exemplary sequence rearrangements can also result from a break and subsequent inter- or intra-chromosomal translocation. In particular embodiments, a sequence rearrangement includes a break in a sequence region from about 56,498,495 to about 59,005,059 of chromosome 1, and translocation to chromosome 3, in a sequence region from about 150,104,752 to about 150,651,284; a break in a sequence region from about 56,498,495 to about 59,005,059 of chromosome 1, and translocation to chromosome 4, in a sequence region from about 123,278,910 to about 125,141,341; a break in a sequence region from about 56,498,495 to about 59,005,059 of chromosome 1, and translocation to chromosome 10, in a sequence region from about 21,581,611 to about 22,244,164; a break in a sequence region from about 56,498,495 to about 59,005,059 of chromosome 1, and translocation to chromosome 11, in a sequence region from about 18,339,189 to about 18,766,440; a break in a sequence region from about 79,177,716 to about 84,414,777 of chromosome 1, and translocation to chromosome 1, in a sequence region from about 56,498,495 to about 59,005,059; a break in a sequence region from about 79,177,716 to about 84,414,777 of chromosome 1, and translocation to chromosome 10, in a sequence region from about 24,328,653 to about 25,616,569; a break in a sequence region from about 79,177,716 to about 84,414,777 of chromosome 1, and translocation to chromosome 10, in a sequence region from about 26,780,251 to about 27,150,556; a break in a sequence region from about 5,174,608 to about 9,099,558 of chromosome 2, and translocation to chromosome 6, in a sequence region from about 12,953,556 to about 13,492,116; a break in a sequence region from about 5,174,608 to about 9,099,558 of chromosome 2, and translocation to chromosome 14, in a sequence region from about 74,999,855 to about 77,279,911; a break in a sequence region from about 57,825,183 to about 61,899,453 of chromosome 2, and translocation to chromosome 1, in a sequence region from about 182,351,950 to about 182,647,216; a break in a sequence region from about 72,517,657 to about 74,474,129 of chromosome 3, and translocation to chromosome 16, in a sequence region from about 4,902,761 to about 5,140,847; a break in a sequence region from about 156,565,132 to about 158,632,403 of chromosome 5, and translocation to chromosome 6, in a sequence region from about 12,953,556 to about 13,492,116; a break in a sequence region from about 7,047,303 to about 9,164,260 of chromosome 6, and translocation to chromosome 5, in a sequence region from about 127,469,416 to about 128,152,120; a break in a sequence region from about 155,264,117 to about 157,210,205 of chromosome 7, and translocation to chromosome 2, in a sequence region from about 204,546,848 to about 205,747,855; a break in a sequence region from about 92,587,940 to about 94,938,420 of chromosome 8, and translocation to chromosome 8, in a sequence region from about 95,158,106 to about 97,246,188; a break in a sequence region from about 92,587,940 to about 94,938,420 of chromosome 8, and translocation to chromosome 8, in a sequence region from about 100,204,991 to about 101,300,870; a break in a sequence region from about 92,587,940 to about 94,938,420 of chromosome 8, and translocation to chromosome 8, in a sequence region from about 73,524,706 to about 74,020,731; a break in a sequence region from about 30,351,542 to about 32,975,808 of chromosome 11, and translocation to chromosome 11, in a sequence region from about 38,573,713 to about 38,786,646; a break in a sequence region from about 41,040,453 to about 45,974,198 of chromosome 12, and translocation to chromosome 12, in a sequence region from about 21,680,651 to about 25,047,423; a break in a sequence region from about 53,236,066 to about 55,250,543 of chromosome 13, and translocation to chromosome 13, in a sequence region from about 61,279,987 to about 61,544,511; a break in a sequence region from about 58,902,901 to about 61,141,887 of chromosome 13, and translocation to chromosome 5, in a sequence region from about 131,975,089 to about 132,437,799; a break in a sequence region from about 94,878,945 to about 99,073,175 of chromosome 15, and translocation to chromosome 6, in a sequence region from about 97,236,933 to about 100,229,929; a break in a sequence region from about 6,703,581 to about 9,024,395 of chromosome 16, and translocation to chromosome 16, in a sequence region from about 6,186,373 to about 6,467,032; a break in a sequence region from about 18,877,624 to about 23,308,408 of chromosome 18, and translocation to chromosome 18, in a sequence region from about 31,179,004 to about 31,808,361; a break in a sequence region from about 18,877,624 to about 23,308,408 of chromosome 18, and translocation to chromosome 18, in a sequence region from about 68,968,542 to about 69,294,308; a break in a sequence region from about 18,877,624 to about 23,308,408 of chromosome 18, and translocation to chromosome 20, in a sequence region from about 30,073,091 to about 31,440,748; a break in a sequence region from about 30,115,800 to about 33,770,238 of chromosome 19, and translocation to chromosome 19, in a sequence region from about 29,570,255 to about 30,082,475. The numerical coordinates for genomic synteny block sequence are as defined in the Human Genome Reference Consortium, Version GRCh37.

In accordance with the invention, there are still further provided kits and arrays that include nucleic acid probes or primers, such as probes and primers useful for detecting the presence or absence of a chromosomal sequence rearrangement within genomic synteny block sequences. In one embodiment, a kit or array includes one or more nucleic acid probes, wherein each probe hybridizes to a nucleic acid including a chromosomal sequence rearrangement within one or more genomic synteny block sequences (e.g., a sequence selected from: chromosome 1, in a sequence from about 79,177,716 to about 84,414,777; chromosome 1, in a sequence region from about 56,498,495 to about 59,005,059; chromosome 2, in a sequence region from about 5,174,608 to about 9,099,558; chromosome 2, in a sequence region from about 57,825,183 to about 61,899,453; chromosome 3, in a sequence region from about 72,517,657 to about 74,474,129; chromosome 5, in a sequence region from about 156,565,132 to about 158,632,403; chromosome 6, in a sequence region from about 7,047,303 to about 9,164,260; chromosome 7, in a sequence region from about 155,264,117 to about 157,210,205; chromosome 8, in a sequence region from about 92,587,940 to about 94,938,420; chromosome 11, in a sequence region from about 30,351,542 to about 32,975,808; chromosome 12, in a sequence region from about 41,040,453 to about 45,974,198; chromosome 13, in a sequence region from about 53,236,066 to about 55,250,543; chromosome 13, in a sequence region from about 58,902,901 to about 61,141,887; chromosome 15, in a sequence region from about 94,878,945 to about 99,073,175; chromosome 16, in a sequence region from about 6,703,581 to about 9,024,395; chromosome 18, in a sequence region from about 18,877,624 to about 23,308,408; chromosome 19, in a sequence region from about 30,115,800 to about 33,770,238, and the sequence rearrangement is all or a portion of any of the foregoing genomic synteny block sequences), wherein at least one of the probes can detect the presence of a foregoing chromosomal sequence rearrangement.

In another embodiment, a kit or array includes one or more nucleic acid probes, wherein each probe hybridizes to a nucleic acid including a chromosome sequence break or translocation (e.g., a sequence break or translocation is any of break in a sequence region from about 56,498,495 to about 59,005,059 of chromosome 1, and translocation to chromosome 3, in a sequence region from about 150,104,752 to about 150,651,284; a break in a sequence region from about 56,498,495 to about 59,005,059 of chromosome 1, and translocation to chromosome 4, in a sequence region from about 123,278,910 to about 125,141,341; a break in a sequence region from about 56,498,495 to about 59,005,059 of chromosome 1, and translocation to chromosome 10, in a sequence region from about 21,581,611 to about 22,244,164; a break in a sequence region from about 56,498,495 to about 59,005,059 of chromosome 1, and translocation to chromosome 11, in a sequence region from about 18,339,189 to about 18,766,440; a break in a sequence region from about 79,177,716 to about 84,414,777 of chromosome 1, and translocation to chromosome 1, in a sequence region from about 56,498,495 to about 59,005,059; a break in a sequence region from about 79,177,716 to about 84,414,777 of chromosome 1, and translocation to chromosome 10, in a sequence region from about 24,328,653 to about 25,616,569; a break in a sequence region from about 79,177,716 to about 84,414,777 of chromosome 1, and translocation to chromosome 10, in a sequence region from about 26,780,251 to about 27,150,556; a break in a sequence region from about 5,174,608 to about 9,099,558 of chromosome 2, and translocation to chromosome 6, in a sequence region from about 12,953,556 to about 13,492,116; a break in a sequence region from about 5,174,608 to about 9,099,558 of chromosome 2, and translocation to chromosome 14, in a sequence region from about 74,999,855 to about 77,279,911; a break in a sequence region from about 57,825,183 to about 61,899,453 of chromosome 2, and translocation to chromosome 1, in a sequence region from about 182,351,950 to about 182,647,216; a break in a sequence region from about 72,517,657 to about 74,474,129 of chromosome 3, and translocation to chromosome 16, in a sequence region from about 4,902,761 to about 5,140,847; a break in a sequence region from about 156,565,132 to about 158,632,403 of chromosome 5, and translocation to chromosome 6, in a sequence region from about 12,953,556 to about 13,492,116; a break in a sequence region from about 7,047,303 to about 9,164,260 of chromosome 6, and translocation to chromosome 5, in a sequence region from about 127,469,416 to about 128,152,120; a break in a sequence region from about 155,264,117 to about 157,210,205 of chromosome 7, and translocation to chromosome 2, in a sequence region from about 204,546,848 to about 205,747,855; a break in a sequence region from about 92,587,940 to about 94,938,420 of chromosome 8, and translocation to chromosome 8, in a sequence region from about 95,158,106 to about 97,246,188; a break in a sequence region from about 92,587,940 to about 94,938,420 of chromosome 8, and translocation to chromosome 8, in a sequence region from about 100,204,991 to about 101,300,870; a break in a sequence region from about 92,587,940 to about 94,938,420 of chromosome 8, and translocation to chromosome 8, in a sequence region from about 73,524,706 to about 74,020,731; a break in a sequence region from about 30,351,542 to about 32,975,808 of chromosome 11, and translocation to chromosome 11, in a sequence region from about 38,573,713 to about 38,786,646; a break in a sequence region from about 41,040,453 to about 45,974,198 of chromosome 12, and translocation to chromosome 12, in a sequence region from about 21,680,651 to about 25,047,423; a break in a sequence region from about 53,236,066 to about 55,250,543 of chromosome 13, and translocation to chromosome 13, in a sequence region from about 61,279,987 to about 61,544,511; a break in a sequence region from about 58,902,901 to about 61,141,887 of chromosome 13, and translocation to chromosome 5, in a sequence region from about 131,975,089 to about 132,437,799; a break in a sequence region from about 94,878,945 to about 99,073,175 of chromosome 15, and translocation to chromosome 6, in a sequence region from about 97,236,933 to about 100,229,929; a break in a sequence region from about 6,703,581 to about 9,024,395 of chromosome 16, and translocation to chromosome 16, in a sequence region from about 6,186,373 to about 6,467,032; a break in a sequence region from about 18,877,624 to about 23,308,408 of chromosome 18, and translocation to chromosome 18, in a sequence region from about 31,179,004 to about 31,808,361; a break in a sequence region from about 18,877,624 to about 23,308,408 of chromosome 18, and translocation to chromosome 18, in a sequence region from about 68,968,542 to about 69,294,308; a break in a sequence region from about 18,877,624 to about 23,308,408 of chromosome 18, and translocation to chromosome 20, in a sequence region from about 30,073,091 to about 31,440,748; a break in a sequence region from about 30,115,800 to about 33,770,238 of chromosome 19, and translocation to chromosome 19, in a sequence region from about 29,570,255 to about 30,082,475), wherein at least one of the probes can detect the presence of one of the foregoing sequence translocations.

In embodiments with primers, such as kits and arrays, typically the primers are primer pairs, where each primer pair is oppositely oriented to each other, and each of the primer pairs hybridizes to a sequence region that includes or flanks a somatic chromosomal rearrangement, or a nucleic acid derived from the somatic chromosomal rearrangement (e.g., one or more rearrangements with a genomic synteny block sequence as set forth herein). Such primers pairs that hybridize to a sequence region that includes or flanks a somatic chromosomal rearrangement, are useful for detecting the presence or absence of somatic chromosomal rearrangements, in accordance with the invention methods, systems, databases, kits, etc.

### DESCRIPTION OF DRAWINGS

FIG. 1 shows a representative map of a chromosomal sequence rearrangement, a sequence translocation of a species conserved sequence region.

FIG. 2 shows a sequence translocation of dense conserved non-coding DNA from a 4 Mb long syntenic segment on chromosome 2 to chromosome 1, which is found in the breast cancer cell PD3664a. The 4 Mb segment is dense in conserved non-coding DNA and is preserved across multiple species (Human, Mouse and Chicken).

FIG. 3 shows a sequence translocation of dense conserved non-coding DNA from a 2 Mb long conserved segment on chromosome 3 to chromosome 16 in front of PPL, a gene regulating cell growth. This translocation is found in breast cancer cell PD3668a.

FIG. 4 shows a sequence translocation of dense conserved non-coding DNA from a 2 Mb long conserved segment on chromosome 7 that contains LMBR1 and SHH, two genes involved in development of the embryo limbs. The non-coding DNA is translocated in front of ICOS, a gene reported to regulate cell proliferation. This translocation is found in breast cancer cell PD3687a.

FIG. 5 shows a sequence translocation of dense conserved non-coding DNA from a 2 Mb long conserved segment on chromosome 6 that contain BMP6, a gene involved in embryogenesis. The non-coding DNA is translocated in front of several genes on chromosome 5 which function is not clearly known. This translocation is found in breast cancer cell PD3690a.

FIG. 6 shows an example of a recurrent sequence translocation: 3 translocations found in 2 different cancer cells, 1 Colon and 1 Breast translocate dense non-coding DNA from the same 4 mb segment on chromosome 2 that contains the embryonic development genes SOX11 and RNF144A. This translocation may dysregulate the gene TBC1D7, a gene that regulates cell growth. It is also dysregulated by another translocation from a region on chromosome 5 that contains ABAM19 and SOX30, two developmental genes.

FIG. 7 shows an example of sequence translocation recurrence: Several translocations in pancreas and lung translocate the same non-coding regions.

FIG. 8 shows an example of sequence translocation recurrence: Several translocations in pancreas and breast translocate the same non-coding regions on chromosome 18. The non-coding DNA may have been preserved to regulate LAMAS, an embryonic development gene. In this breast cancer cell, it is translocated in front of the cell growth gene ID1.

FIG. 9 shows a system 10, configured to correlate chromosomal sequence rearrangements with the presence of a tumor or cancer, or with an increased risk of tumor or cancer, and/or to identify the presence of a tumor or cancer, or an increased risk of tumor or cancer, in a sample.

FIG. 10 shows a schematic outline of identifying chromosomal sequence rearrangements correlating with the presence of a tumor or cancer, or with an increased risk of tumor or cancer, by using a synteny filter for sequence rearrangements, such as translocations, and recurrence filter for sequence rearrangements that recur in multiple tumors, cancers or different subjects.

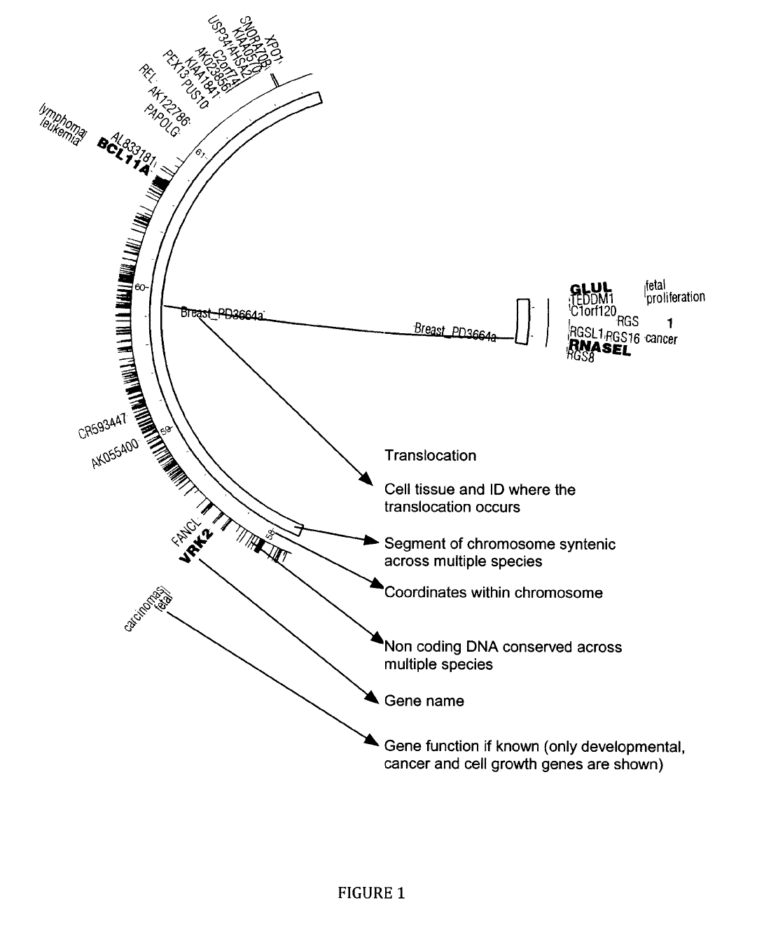

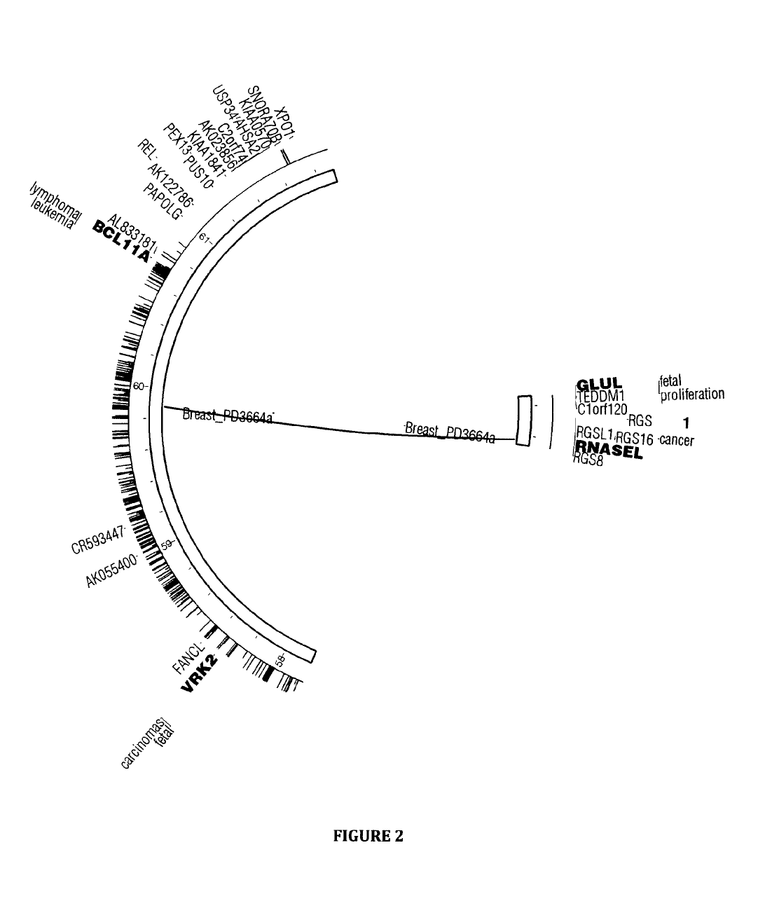

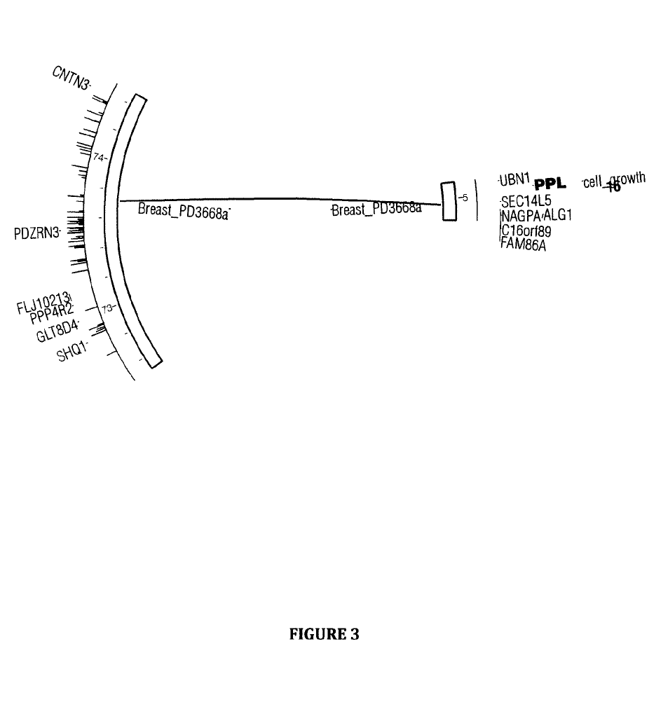

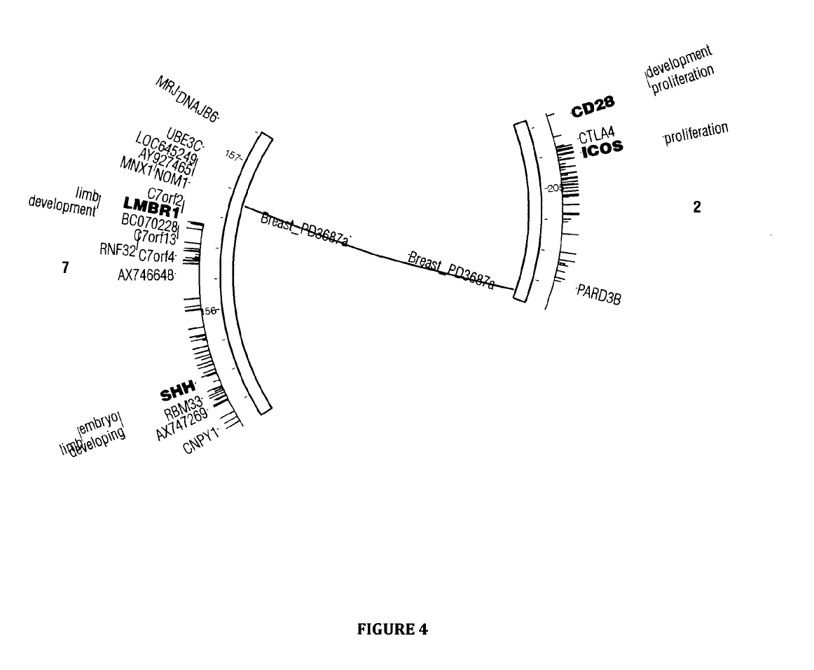

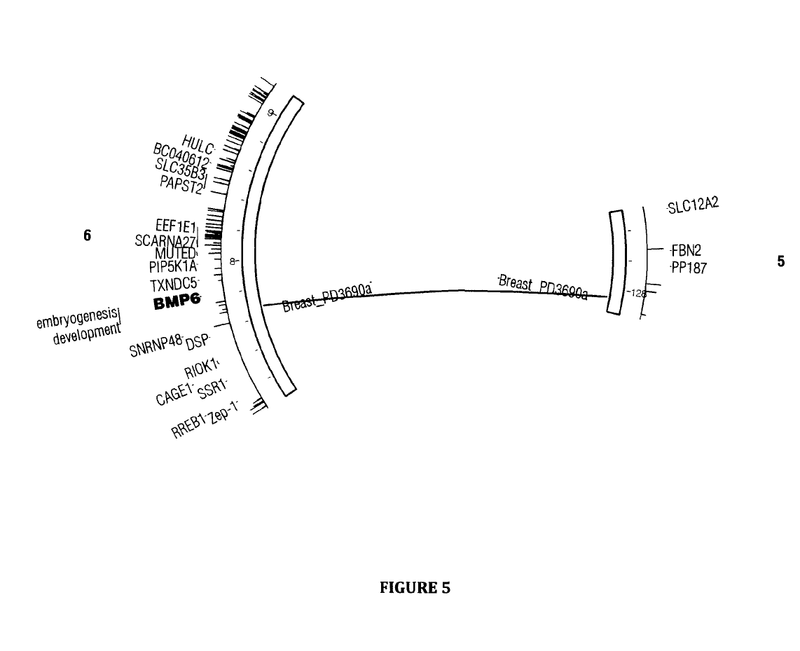

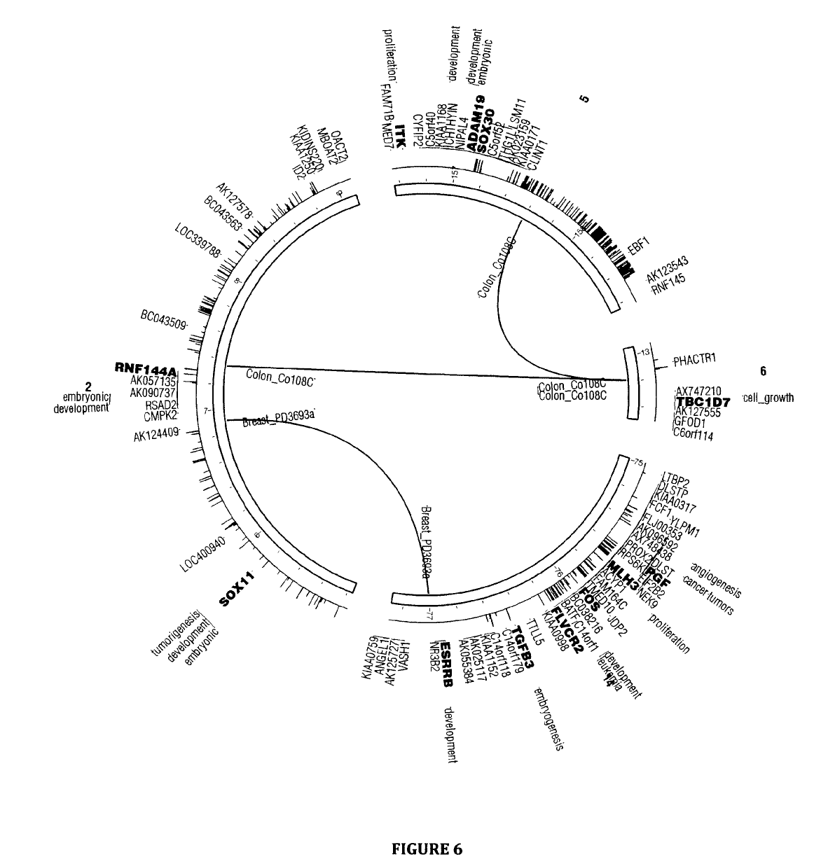

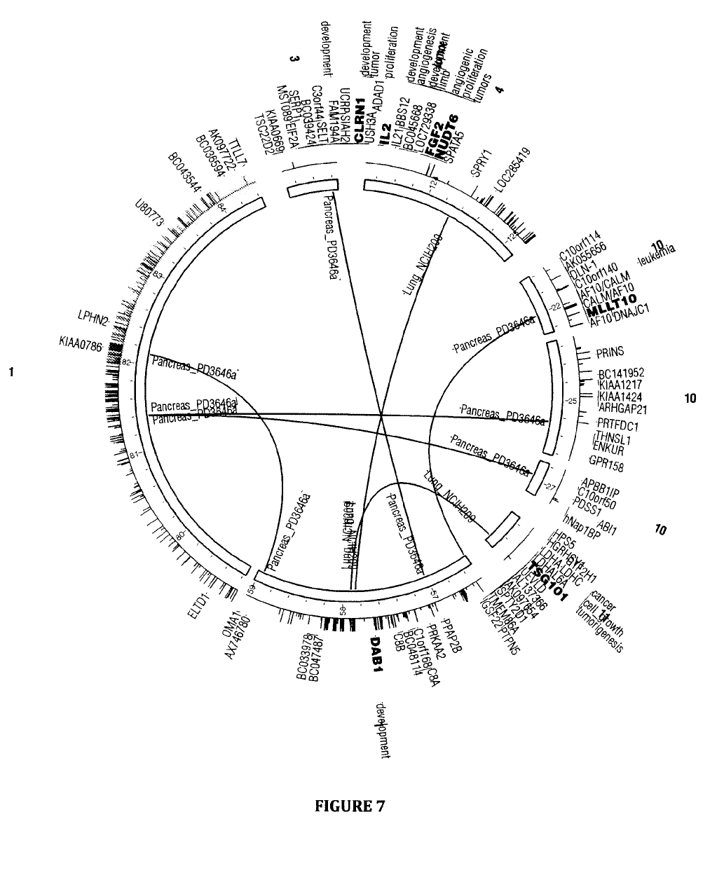

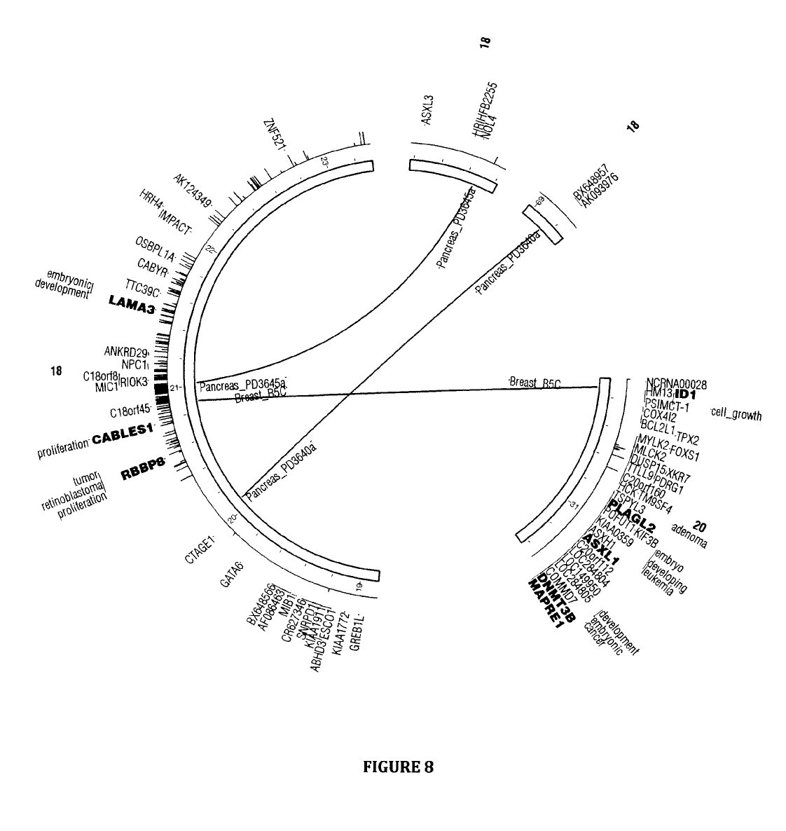

### DETAILED DESCRIPTION

The invention relates to somatic chromosomal sequence rearrangements that correlate with an increased risk of or the presence of a tumor or cancer. As disclosed herein, particular somatic chromosomal sequence rearrangements have been identified in various tumors and cancers, including pancreas, lung, breast and colon tumors and cancers. The presence of such somatic chromosomal sequence rearrangements in a subject therefore indicates an increased risk of or the presence of a tumor or cancer. Screening for somatic chromosomal sequence rearrangements, can be used to ascertain or predict the presence or risk of a subject having a tumor or cancer. For example, the presence of one or more somatic chromosomal sequence rearrangements in a sample from a subject can be determined Detection, measurement or analysis of one or more such somatic chromosomal sequence rearrangements predictive of a tumor or cancer provides information as to whether the subject has or is at increased risk of a tumor or cancer.

Accordingly, the invention provides methods for predicting the presence or absence of a tumor or cancer in a subject, and determining the risk of a tumor or cancer in a subject. In one embodiment, genomic nucleic acid of a subject is analyzed (e.g., screened) for the presence or absence of a somatic chromosomal sequence rearrangement predictive of the presence of tumor or cancer or an increased risk of tumor or cancer, where the somatic chromosomal sequence rearrangement is in a species conserved genomic synteny block sequence, and where all or a portion of the species conserved genomic synteny block sequence is structurally rearranged to be in an altered proximity to a protein coding sequence. Presence of the somatic chromosomal sequence rearrangement in a synteny block sequence is predictive of the presence of tumor or cancer in the subject or an increased risk of tumor or cancer in the subject; whereas absence of the somatic chromosomal sequence rearrangement in a synteny block sequence is predictive of the absence of tumor or cancer in the subject or a reduced risk of tumor or cancer in the subject.

Likewise, screening for altered expression of one or more gene expression products (i.e., protein), whose expression is altered as a consequence of a somatic chromosomal sequence rearrangement, such as a rearranged species conserved synteny block sequence, can provide information as to whether the subject has or is at increased risk of a tumor or cancer. Detection, measurement or analysis of one or more such gene expression products can therefore also be used to predict whether the subject has or is at increased risk of a tumor or cancer.

Accordingly, the invention also provides methods for predicting the presence or absence of a tumor or cancer in a subject, and determining the risk of a tumor or cancer in a subject. In one embodiment, expression of a gene coding sequence of a subject is analyzed (e.g., screened) for the presence or absence of altered gene product expression predictive of the presence of tumor or cancer or an increased risk of tumor or cancer, where the gene coding sequence has an altered position due to a somatic chromosomal sequence rearrangement of a species conserved genomic synteny block sequence. Altered expression of the gene coding sequence is predictive of the presence of tumor or cancer in the subject or an increased risk of tumor or cancer in the subject; whereas expression comparable to normal levels expression (e.g., relative to normal counterpart cells) is predictive of the absence of tumor or cancer in the subject or a reduced risk of tumor or cancer in the subject.

Detection, measurement or analysis of one or more such somatic chromosomal sequence rearrangements, or gene expression products, predictive of the presence of a tumor or cancer in a subject can also be used to provide information concerning the status of the tumor or cancer in the subject. Thus, somatic chromosomal sequence rearrangements, or gene expression products, can also be used to monitor regression or progression or worsening (e.g., metastasis) of a tumor or cancer. For example, a decreased quantity of a somatic chromosomal sequence rearrangement of a synteny block sequence in a sample from a subject with a tumor or cancer can indicate regression or improvement of the tumor or cancer. In contrast, an increased quantity of a somatic chromosomal sequence rearrangement of a synteny block sequence in a sample from a subject with a tumor or cancer can indicate progression or worsening (e.g., metastasis) of the tumor or cancer.

Accordingly, the invention also provides methods for monitoring progression or regression of a tumor or cancer in a subject. In one embodiment, genomic nucleic acid of a sample from a subject is analyzed to determine an amount of nucleic acid comprising a somatic chromosomal sequence rearrangement indicative of a tumor or cancer; wherein the somatic chromosomal sequence rearrangement is within a species conserved genomic synteny block sequence. An amount of somatic chromosomal sequence rearrangement in the sample greater as compared to a prior sample indicates increased tumor or cancer load, and likely progression or worsening of the tumor or cancer in the subject. An amount of the somatic chromosomal sequence rearrangement in the sample less as compared to a prior sample indicates reduced tumor or cancer load, and a likely regression of the tumor or cancer in the subject.

Identifying correlations of somatic chromosomal sequence rearrangements, or altered expression of gene expression products, predictive of an increased risk or the presence of a tumor or cancer in a subject can be used to provide information concerning somatic chromosomal sequence rearrangements or altered expression of gene expression products indicative of the presence of a tumor or cancer, or an increased risk of tumor or cancer. Such correlating somatic chromosomal sequence rearrangements, or gene expression products, in turn can be used for the purpose of analyzing samples from subjects for the presence of somatic chromosomal sequence rearrangements, for example, in a genomic synteny block sequence, or altered expression of a gene expression product, in order to ascertain or determine if the subject is at an increased risk or has a tumor or cancer.

Accordingly, the invention further provides methods for identifying somatic chromosomal sequence rearrangements correlating with the presence of a tumor or cancer, or with an increased risk of tumor or cancer. In one embodiment, a method includes analyzing genomic nucleic acid of a sample from a tumor or cancer to determine the presence or absence of a somatic chromosomal sequence rearrangement (e.g., in a genomic synteny block sequence); comparing the somatic chromosomal sequence rearrangement, if present, to a corresponding germline sequence; and repeating the foregoing steps for one or more additional tumor or cancer samples. If the somatic chromosomal sequence rearrangement is recurrent, in other words, occurs in multiple tumor or cancer cell genomic nucleic acid and is absent from a corresponding germline sequence, the somatic chromosomal sequence rearrangement is identified as predictive of the presence of tumor or cancer or an increased risk of tumor or cancer.

Identifying correlations of somatic chromosomal sequence rearrangements (e.g., in a genomic synteny block sequence), or altered expression of a gene expression product, predictive of an increased risk or the presence of a tumor or cancer in a subject can also be used to construct a database or organizational construct. Such databases and organizational constructs can in turn be used for the purpose of analyzing samples from subjects for such somatic chromosomal sequence rearrangements, for example, in a genomic synteny block sequence, or altered expression of a gene expression product, in order to ascertain or determine if the subject is at an increased risk or has a tumor or cancer.

Accordingly, the invention further provides methods of producing databases and organizational constructs having somatic chromosomal sequence rearrangements predictive of the presence of tumor or cancer, or an increased risk of a tumor or cancer. In one embodiment, a method includes analyzing tumor or cancer cell genomic nucleic acid for the presence or absence of a somatic chromosomal sequence rearrangement of a synteny block sequence (e.g., translocation), and comparing the sequence arrangement to a corresponding germline sequence. The presence of the somatic chromosomal sequence rearrangement in the tumor or cancer cell genomic nucleic acid absent from a corresponding germline sequence indicates the somatic chromosomal sequence rearrangement as predictive of the presence of tumor or cancer or an increased risk of tumor or cancer. In particular aspects, methods include recording or storing information concerning the presence or absence of the somatic chromosomal sequence rearrangement that predicts presence of tumor or cancer or an increased risk of tumor or cancer, thereby producing a database or organizational construct comprising a somatic chromosomal sequence rearrangement predictive of the presence of tumor or cancer or an increased risk of tumor or cancer. Optionally, the methods include repeating steps analysis of different tumors or cancers, comparison and recording or storing analysis for somatic chromosomal sequence rearrangements, thereby producing a database or organizational construct comprising somatic chromosomal sequence rearrangements predictive of the presence of tumor or cancer or an increased risk of tumor or cancer.

In various embodiments, a plurality of sample analysis of multiple and/or different tumors or cancers (tumor or cancer types, stages, grades, etc., or tumors or cancers in different subjects) in turn leads to identification of somatic chromosomal sequence rearrangements (such as translocations in synteny block sequences) that are recurrent, i.e., the rearrangement of the somatic chromosomal sequence “recurs” or “appears” in more than one tumor or cancer type, or in different subjects with a tumor or cancer. Due to recurrence of a somatic chromosomal sequence rearrangement in multiple tumor or cancer types and/or in multiple different patients, such recurrent rearrangements, such as translocations in synteny block sequences, are more relevant to development and/or progression of tumors or cancers. Consequently, recurrent somatic chromosomal sequence rearrangements, such as translocations in synteny block sequences, are of particular value in predicting or diagnosing the presence of a tumor or cancer or an increased risk of tumor or cancer in a subject. Accordingly, recurrent somatic chromosomal sequence rearrangements, such as translocations in synteny block sequences, predictive of the presence of tumor or cancer or an increased risk of tumor or cancer in a subject are of particular value in accordance with the invention.

In accordance with the invention, non-limiting examples of sequence regions in which somatic chromosomal sequence rearrangements occur, in all or a part of a genomic synteny block sequence, include: chromosome 1, in a sequence region from about 79,177,716 to about 84,414,777; chromosome 1, in a sequence region from about 56,498,495 to about 59,005,059; chromosome 2, in a sequence region from about 5,174,608 to about 9,099,558; chromosome 2, in a sequence region from about 57,825,183 to about 61,899,453; chromosome 3, in a sequence region from about 72,517,657 to about 74,474,129; chromosome 5, in a sequence region from about 156,565,132 to about 158,632,403; chromosome 6, in a sequence region from about 7,047,303 to about 9,164,260; chromosome 7, in a sequence region from about 155,264,117 to about 157,210,205; chromosome 8, in a sequence region from about 92,587,940 to about 94,938,420; chromosome 11, in a sequence region from about 30,351,542 to about 32,975,808; chromosome 12, in a sequence region from about 41,040,453 to about 45,974,198; chromosome 13, in a sequence region from about 53,236,066 to about 55,250,543; chromosome 13, in a sequence region from about 58,902,901 to about 61,141,887; chromosome 15, in a sequence region from about 94,878,945 to about 99,073,175; chromosome 16, in a sequence region from about 6,703,581 to about 9,024,395; chromosome 18, in a sequence region from about 18,877,624 to about 23,308,408; chromosome 19, in a sequence region from about 30,115,800 to about 33,770,238, of all or a part of any of the foregoing genomic synteny block sequence regions. Coordinates of such sequence regions are as defined in the Human Genome Reference Consortium, Version GRCh37.

As used herein, the terms “neoplasia” and “tumor” refer to a cell or population of cells whose growth, proliferation or survival is greater than growth, proliferation or survival of a normal counterpart cell, e.g. a cell proliferative or differentiative disorder. A tumor is a neoplasia that has formed a distinct mass or growth. A “cancer” or “malignancy” refers to a neoplasia or tumor that can invade adjacent spaces, tissues or organs. A “metastasis” refers to a neoplasia, tumor, cancer or malignancy that has disseminated or spread from its primary site to one or more secondary sites, locations or regions within the subject, in which the sites, locations or regions are distinct from the primary tumor or cancer.

Neoplastic, tumor, cancer and malignant cells (metastatic or non-metastatic) include dormant or residual neoplastic, tumor, cancer and malignant cells. Such cells typically consist of remnant tumor cells that are not dividing (G0-G1 arrest). These cells can persist in a primary site or as disseminated neoplastic, tumor, cancer or malignant cells as a residual disease. These dormant neoplastic, tumor, cancer or malignant cells remain asymptomatic, but can develop severe symptoms and cause death once these dormant cells proliferate.

In accordance with the invention, neoplastic, tumor, cancer and malignant cells include solid and liquid neoplasias, tumors, cancers and malignancies. Metastatic and non-metastatic tumors, cancers, malignancies or neoplasias may be in any stage, e.g., early or advanced, such as a stage I, II, III, IV or V tumor or cancer. The metastatic or non-metastatic tumor, cancer, malignancy or neoplasia may have been subject to a prior treatment or be stabilized (non-progressing) or in remission, or progressing or worsening.

Neoplasias, tumors, cancers and malignancies include “solid” tumors and cancers, which refers to cancer, neoplasia or metastasis that typically aggregates together and forms a mass. Specific examples include carcinomas (which refer to malignancies of epithelial or endocrine tissue) and sarcomas (which refer to malignant tumors of mesenchymal cell origin). Particular non-limiting examples of neoplasias, tumors, cancers and malignancies include pancreas, lung, colon, and breast tumors and cancers.

As used herein, the term “genomic sequence rearrangement” means a physical or structural change in a chromosome (nucleotide) sequence of a cell that is not normally present in normal cells. The change can result in an increase or decrease of the number of one or more particular nucleotide sequences or sequence segments (elements). A genomic sequence rearrangement can in turn lead to a change in expression (increase or decrease) of a gene coding sequence due to a change to the sequence and/or a change in position or sequence of a regulatory region or sequence in relationship to the gene coding sequence, such as a sequence that affects cell proliferation, differentiation, cell survival or cell death/apoptosis. As an example, in leukemia a Philadelphia translocation fuses BCR and ABL, creating a new oncogene BCR-ABL, which is a hyperactive kinase that activates a pathway that results in abnormally high cell proliferation.

Non-limiting examples of physical or structural chromosomal sequence changes include genomic sequence deletions or additions, tandem or inverted sequence repeats and duplications, and inter-chromosomal or intra-chromosomal sequence translocations. As used herein, the term “chromosomal sequence translocation” refers to a chromosome sequence that has been rearranged within the same chromosome (the sequence moves from one position to another in the same chromosome) or with a different chromosome (the sequence moves from one chromosome to a different chromosome). A chromosomal sequence translocation can be reciprocal or non-reciprocal. A reciprocal translocation of a sequence from one chromosome to a different chromosome can be balanced, where the sequence is exchanged with the same length of sequence from the different chromosome, or non-balanced where different sequence lengths are exchanged between the two different chromosomes.

For a “genomic sequence rearrangement” the number of nucleotides that are rearranged can be as few as 2-5, or 5-10, but typically the length of the sequence rearrangements are larger. Non-limiting examples of sequence rearrangement lengths (e.g., deletions, additions, tandem or inverted repeats, translocations, etc.) include, but are not limited to, 10-20, 20-50, 50-100, 100-500, 500-1,000, 1,000-5,000, 5,000-10,000, 10,000-50,000, 50,000-100,000, 100,000-250,000, 250,000-500,000, 500,000-1,000,000, 1,000,000-2,000,000, 2,000,000-5,000,000, 5,000,000-10,000,000, 10,000,000-20,000,000, or more nucleotide sequences. Such sequences can be conveniently referred to as sequence elements or segments, which elements or segments comprise a given length of nucleotides.

Non-limiting examples of sequence translocations include: chromosome 1, in a sequence region from about 56,498,495 to about 59,005,059; chromosome 1, in a sequence region from about 182,351,950 to about 182,647,216; chromosome 2, in a sequence region from about 204,546,848 to about 205,747,855; chromosome 3, in a sequence region from about 150,104,752 to about 150,651,284; chromosome 4, in a sequence region from about 123,278,910 to about 125,141,341; chromosome 5, in a sequence region from about 127,469,416 to about 128,152,120; chromosome 5, in a sequence region from about 131,975,089 to about 132,437,799; chromosome 6, in a sequence region from about 12,953,556 to about 13,492,116; chromosome 6, in a sequence region from about 97,236,933 to about 100,229,929; chromosome 8, in a sequence region from about 95,158,106 to about 97,246,188; chromosome 8, in a sequence region from about 100,204,991 to about 101,300,870; chromosome 8, in a sequence region from about 73,524,706 to about 74,020,731; chromosome 10, in a sequence region from about 24,328,653 to about 25,616,569; chromosome 10, in a sequence region from about 26,780,251 to about 27,150,556; chromosome 10, in a sequence region from about 21,581,611 to about 22,244,164; chromosome 11, in a sequence region from about 18,339,189 to about 18,766,440; chromosome 11, in a sequence region from about 38,573,713 to about 38,786,646; chromosome 12, in a sequence region from about 21,680,651 to about 25,047,423; chromosome 13, in a sequence region from about 61,279,987 to about 61,544,511; chromosome 14, in a sequence region from about 74,999,855 to about 77,279,911; chromosome 16, in a sequence region from about 4,902,761 to about 5,140,847; chromosome 16, in a sequence region from about 6,186,373 to about 6,467,032; chromosome 18, in a sequence region from about 31,179,004 to about 31,808,361; chromosome 18, in a sequence region from about 68,968,542 to about 69,294,308; chromosome 19, in a sequence region from about 29,570,255 to about 30,082,475; chromosome 20, in a sequence region from about 30,073,091 to about 31,440,748. Coordinates for the foregoing sequence regions are as defined in the Human Genome Reference Consortium, Version GRCh37.

Exemplary “genomic sequence rearrangements” can occur in a species conserved genomic sequence region, such as a synteny block sequence. As used herein, a “genomic synteny block sequence” is a genomic sequence region that is conserved between two or more species of animal (e.g., typically vertebrates, such as human, mouse and/or chicken). In a particular embodiment, the species are human, mouse and/or chicken, i.e. the sequences are conserved among two or more of these species.

Typically, “genomic synteny block sequences” can include non-coding sequences, segments or elements and/or gene coding sequence, segment or element (e.g., exons or open reading frames). As used herein a “non-coding sequence, segment or element” refers to a nucleotide sequence that does not appear to be transcribed and translated into an amino acid sequence. As used herein a “coding sequence, segment or element” or “gene coding sequence, segment or element” refers to an open reading frame or exon that codes for a specific amino acid sequence. Such coding sequences, segments or elements for amino acid sequences may or may not be transcribed or translated due to cell or tissue type, differentiation stage, regulatory environment, etc.

Typically, over a given portion of the genomic synteny block sequence, a plurality of non-coding sequences, segments or elements, and/or gene coding sequences segments or elements (if present) are in the same order along the chromosome—that is, the position of a non-coding sequence, segment or element or a gene coding sequence, segment or element along the chromosome is conserved (maintained) between species. A “genomic synteny block sequence” conserved among various species of animals (e.g., vertebrates), when used in reference to a genomic sequence therefore includes a plurality of non-coding sequences, segments or elements over a given sequence length, sharing the same order over a given sequence length, and/or, if present, a plurality of gene coding sequences, segments or elements (i.e., open reading frames or exons that encode protein) sharing the same order over a given sequence length. The number of non-coding or gene coding sequences, segments or elements that have the same order depends upon the genomic synteny block sequence, and can range, for example, from 2-10, 10-20, 20-50, 50-100, 100-500, 500-1,000, 1,000-5,000, 5,000-10,000, 10,000-25,000, 25,000-50,000, 50,000-100,000, or more segments or elements within a given genomic synteny block sequence, or any numerical value or range within or encompassing such lengths.

In various embodiments, a genomic synteny block sequence is greater than 500,000 nucleotides, or greater than 1 million nucleotides, more typically, greater than 1.5 million nucleotides (e.g., 1.6, 1.7, 1.8, 1.9 million nucleotides, or greater), or greater than 2 million nucleotides (e.g., 3, 4 or 5 million nucleotides, or greater), such as 5 million or more nucleotides (e.g., 6, 7, 8, 9, or 10). Within such genomic synteny block sequences, typically there are at least 5, 10, 15, 20 or more (e.g., 21, 22, 23, 24), 25 or more (e.g., 26, 27, 28, 29, 30), or more, species conserved non-coding “segments” or “elements” for every 1 million nucleotides. Accordingly, a genomic synteny block sequence is composed of “segments” or “elements,” with varying numbers and lengths of non-coding and/or coding nucleotides.

As used herein, the term “segment” or “element” when used in reference to a genomic synteny block sequence refers to a stretch of contiguous nucleotides within the genomic synteny block sequence that is a discrete sequence, such as stretches of non-coding sequences with know or unknown function, non-coding sequences that flank developmental gene coding sequences, non-coding intervening sequence, or an open reading frame or exon of a gene coding sequence. The length of non-coding and gene coding segments or elements can vary significantly, for example, such non-coding segments or elements can be from about 10-20, 20-30, 30-50, 50-100, 100-150, 150-200, 200-250, 250-300, 300400, 400-500, 500-1000, 1000-2000, 2,000-5,000, 5,000-10,000, 10,000-25,000, 25,000-50,000 nucleotides, or any numerical value or range within or encompassing such lengths. Typically, gene coding segments or elements are in a range of from about 10-20, 20-30, 30-50, 50-100, 100-150, 150-200, 200-250, 250-300, 300-400, 400-500, 500-1000, 1000-2000, 2,000-5,000, 5,000-10,000, 10,000-25,000, 25,000-50,000 nucleotides, or any numerical value or range within or encompassing such lengths.

Non-coding and gene coding sequences, segments or elements within a genomic synteny block sequence can have varied ratios or density of non-coding to gene coding. For example, a genomic synteny block sequence may have a higher density or ratio of non-coding sequence regions, segments or elements compared to gene (protein) coding sequence regions, sequences or elements (i.e., open reading frames or exons that encode protein sequences). In various embodiments, a genomic synteny block sequence has a density (or ratio) of non-coding segments or elements of at least 3 (3, 4, 5, 6, 7, 8, 9, 10-20, 20-50, 50-100, or 100-150 or more) to every one gene coding segment or element (exon or open reading frame). In further embodiments, density (or ratio) of gene coding sequence segments or elements (exons or open reading frames) is 1.0 or less (e.g., 0.90, 0.80, 0.70, 0.60, 0.50, 0.40, 0.30, 0.20, 0.10, or less), per 50,000 base pairs. In additional embodiments, a genomic synteny block sequence has non-coding genomic segments or elements of at least 5 (5, 6, 7, 8, 9, 10-20, 20-50, 50-100, or 100-150 or more) to every one gene coding segment or element (exon or open reading frame), and a density (or ratio) of gene coding sequence segments or elements of 0.50 or less (0.50, 0.40, 0.30, 0.20, 0.10, or less) per 100,000 base pairs. Average density (or ratio) of non-coding segments or elements is within about 10-50 non-coding segments or elements per one million base pairs, within a genomic synteny block sequence.

Typically, “genomic synteny block sequences” exhibit inter-species nucleotide sequence conservation with respect to the sequence identity or homology of the non-coding and/or coding sequences, segments or elements that comprise a genomic synteny block sequence between the comparison species of (e.g., animals, such as between vertebrates, human, mouse and/or chicken). Such inter-species conservation or nucleotide sequence identity (homology) can be represented by percentage of sequence identity. Accordingly in various embodiments, species nucleotide sequence conservation, as represented by nucleotide sequence identity, can be as little as 50% or more, or 60%, or more, or be greater, for example, 70% or more identity (e.g., 70%-80%, 80%-90%, 90%-95%, or more than 95%) of sequences, segments or elements within a genomic synteny block sequence shared between the comparison species.

As disclosed herein, genomic sequence conservation or sequence identity among species sequences, segments or elements can be represented by the extent to which positions of analogous sequences, segments or elements (typically non-coding sequences, segments or elements or gene coding sequences, segments or elements such as open reading frames or exons) in the compared genomic sequences are in the same order, or are identical at the nucleotide sequence level. Accordingly, in one embodiment, over a comparison region between species, a non-coding or a gene coding sequence, segment or element is in the same order within an inter-species conserved genomic synteny block sequence. In another embodiment, 50%, 60%, 70% or more (e.g., 70%-80%, 80%-90%, 90%-95%, or more than 95%) of the non-coding or gene coding sequences, segments or elements within the genomic synteny block sequence are in the same order between the compared species. For purposes of further defining a comparison region, such a region can be, without limitation, over 10-50, 50-100, 100 or more (e.g., 100-1,000), 1,000 or more (e.g., 1,000-5,000), 5,000 or more (e.g., 5,000-10,000), 10,000 or more (e.g., 10,000-25,000), 25,000 or more (e.g., 25,000-50,000), 50,000 or more (e.g., 50,000-100,000), or 100,000 or more (e.g., 100,000 or more, 200,000 or more, 300,000 or more, 400,000 or more, or 500,000 or more, e.g., 100,000-1,000,000), or 1,000,000 or more (e.g., 1,000,000-10,000,000) nucleotides in length.

As disclosed herein, sequence conservation or nucleotide sequence identity can extend over a given length of contiguous nucleotides, segments or elements of non-coding or gene coding segments or elements within the genomic synteny block sequences. In particular embodiments, the length of conservation/identity, is measured between 10-50, 50-100, or over 100 or more (e.g., 100-1,000), 1,000 or more (e.g., 1,000-5,000), 5,000 or more (e.g., 5,000-10,000), 10,000 or more (e.g., 10,000-25,000), 25,000 or more (e.g., 25,000-50,000), 50,000 or more (e.g., 50,000-100,000), or 100,000 or more (e.g., 100,000 or more, 200,000 or more, 300,000 or more, 400,000 or more, or 500,000 or more, e.g., 100,000-1,000,000), or 1,000,000 or more (e.g., 1,000,000-10,000,000), base pairs.

Accordingly, inter-species conservation can be reflected by the order of non-coding and/or gene coding sequences, segments or elements—such sequences, segments or elements in the same order/position along the chromosomes between the species indicative of a genomic synteny block sequence, or by a percentage of nucleotide sequence identity along a given sequence, segment or element, of one or more sequences, segments or elements, in a genomic synteny block sequence. Also, inter-species conservation can be a combination of the position (order) of non-coding sequences, segments or elements, or gene coding sequences shared between the species, and a percentage of nucleotide sequence identity along a given sequence, segment or element, of one or more sequences, segments or elements in a genomic synteny block sequence.

Non-limiting examples of inter-chromosomal and intra-chromosomal sequence translocations that occur in include: a break in a sequence region from about 56,498,495 to about 59,005,059 of chromosome 1, and translocation to chromosome 3, in a sequence region from about 150,104,752 to about 150,651,284; a break in a sequence region from about 56,498,495 to about 59,005,059 of chromosome 1, and translocation to chromosome 4, in a sequence region from about 123,278,910 to about 125,141,341; a break in a sequence region from about 56,498,495 to about 59,005,059 of chromosome 1, and translocation to chromosome 10, in a sequence region from about 21,581,611 to about 22,244,164; a break in a sequence region from about 56,498,495 to about 59,005,059 of chromosome 1, and translocation to chromosome 11, in a sequence region from about 18,339,189 to about 18,766,440; a break in a sequence region from about 79,177,716 to about 84,414,777 of chromosome 1, and translocation to chromosome 1, in a sequence region from about 56,498,495 to about 59,005,059; a break in a sequence region from about 79,177,716 to about 84,414,777 of chromosome 1, and translocation to chromosome 10, in a sequence region from about 24,328,653 to about 25,616,569; a break in a sequence region from about 79,177,716 to about 84,414,777 of chromosome 1, and translocation to chromosome 10, in a sequence region from about 26,780,251 to about 27,150,556; a break in a sequence region from about 5,174,608 to about 9,099,558 of chromosome 2, and translocation to chromosome 6, in a sequence region from about 12,953,556 to about 13,492,116; a break in a sequence region from about 5,174,608 to about 9,099,558 of chromosome 2, and translocation to chromosome 14, in a sequence region from about 74,999,855 to about 77,279,911; a break in a sequence region from about 57,825,183 to about 61,899,453 of chromosome 2, and translocation to chromosome 1, in a sequence region from about 182,351,950 to about 182,647,216; a break in a sequence region from about 72,517,657 to about 74,474,129 of chromosome 3, and translocation to chromosome 16, in a sequence region from about 4,902,761 to about 5,140,847; a break in a sequence region from about 156,565,132 to about 158,632,403 of chromosome 5, and translocation to chromosome 6, in a sequence region from about 12,953,556 to about 13,492,116; a break in a sequence region from about 7,047,303 to about 9,164,260 of chromosome 6, and translocation to chromosome 5, in a sequence region from about 127,469,416 to about 128,152,120; a break in a sequence region from about 155,264,117 to about 157,210,205 of chromosome 7, and translocation to chromosome 2, in a sequence region from about 204,546,848 to about 205,747,855; a break in a sequence region from about 92,587,940 to about 94,938,420 of chromosome 8, and translocation to chromosome 8, in a sequence region from about 95,158,106 to about 97,246,188; a break in a sequence region from about 92,587,940 to about 94,938,420 of chromosome 8, and translocation to chromosome 8, in a sequence region from about 100,204,991 to about 101,300,870; a break in a sequence region from about 92,587,940 to about 94,938,420 of chromosome 8, and translocation to chromosome 8, in a sequence region from about 73,524,706 to about 74,020,731; a break in a sequence region from about 30,351,542 to about 32,975,808 of chromosome 11, and translocation to chromosome 11, in a sequence region from about 38,573,713 to about 38,786,646; a break in a sequence region from about 41,040,453 to about 45,974,198 of chromosome 12, and translocation to chromosome 12, in a sequence region from about 21,680,651 to about 25,047,423; a break in a sequence region from about 53,236,066 to about 55,250,543 of chromosome 13, and translocation to chromosome 13, in a sequence region from about 61,279,987 to about 61,544,511; a break in a sequence region from about 58,902,901 to about 61,141,887 of chromosome 13, and translocation to chromosome 5, in a sequence region from about 131,975,089 to about 132,437,799; a break in a sequence region from about 94,878,945 to about 99,073,175 of chromosome 15, and translocation to chromosome 6, in a sequence region from about 97,236,933 to about 100,229,929; a break in a sequence region from about 6,703,581 to about 9,024,395 of chromosome 16, and translocation to chromosome 16, in a sequence region from about 6,186,373 to about 6,467,032; a break in a sequence region from about 18,877,624 to about 23,308,408 of chromosome 18, and translocation to chromosome 18, in a sequence region from about 31,179,004 to about 31,808,361; a break in a sequence region from about 18,877,624 to about 23,308,408 of chromosome 18, and translocation to chromosome 18, in a sequence region from about 68,968,542 to about 69,294,308; a break in a sequence region from about 18,877,624 to about 23,308,408 of chromosome 18, and translocation to chromosome 20, in a sequence region from about 30,073,091 to about 31,440,748; a break in a sequence region from about 30,115,800 to about 33,770,238 of chromosome 19, and translocation to chromosome 19, in a sequence region from about 29,570,255 to about 30,082,475. Coordinates for the foregoing sequence regions are as defined in the Human Genome Reference Consortium, Version GRCh37.

Although not wishing to be bound by any particular theory or hypothesis, a somatic chromosomal sequence rearrangement in a genomic synteny block sequence that changes position of the rearranged sequence relative to one or more gene coding sequences (also referred to as a protein coding sequence) such that the position of such sequences relative to each other is abnormal, can lead to altered expression of encoded protein. Such genes coding expression products can include a protein that modulates cell growth, proliferation, differentiation, survival or apoptosis. Such rearrangement of a genomic synteny block sequence that alters expression of a protein that modulates cell growth, proliferation, differentiation, survival or apoptosis is believed to correlate with, and in fact may contribute to, development, progression or worsening (e.g., metastasis) of a tumor or cancer of a tumor or cancer, and hence explain the correlation of a somatic chromosomal sequence rearrangement in a genomic synteny block sequence the increased risk of or the presence of a tumor or cancer.

Accordingly, in various embodiments, somatic chromosomal sequence rearrangements change the position of a non-coding genomic sequence relative to a gene coding sequence (i.e., an exon or a gene that encodes all or a portion of a protein). Such genes can be involved in regulating or modulating cell growth, proliferation, differentiation, survival or apoptosis. For example, such gene coding sequences may be a protein that promotes or induces cell growth, proliferation, angiogenesis or survival, or a protein that reduces or inhibits cell death (apoptosis), growth inhibition, or survival, as such genes predispose or contribute to development or progression (e.g., metastases) of a tumor or cancer. Particular genes, the altered expression of which is believed to correlate with, and in fact may contribute to, development, progression or worsening (e.g., metastasis) of a tumor or cancer are set forth in Table 2.

In various embodiments, representative gene coding sequences of which a rearrangement of a non-coding genomic sequence is believed to lead to an altered position relative to the non-coding sequence include, but are not limited to, ADAM19, ASXL1, BCAT1, BCL11A, BMP6, CABLES1, CCNE1, CCNE2, CD28, CLRN1, CMAS, CNTN1, COX6C, DAB1, DNMT3B, ESRRB, FGF2, FLVCR2, FOS, GDF6, GLUL, ICOS, ID1, IL2, ITK, KIAA1109, LAMA3, LECT1, LMBR1, MAPRE1, MLH3, MLLT10, MPPED2, NELL2, NUDT6, PAX6, PGF, PLAGL2, PPL, RAD50, RAD54B, RBBP8, RCN1, RNASEL, RNF144A, RUNX1T1, SHH, SHROOM1, SOX11, SOX30, SOX5, TBC1D7, TGFB3, TSG101, VPS13B, VRK2, WIT1, and WT1. The complete names of the foregoing gene sequences are listed in Table 2, and genomic nucleotide sequences for each of these genes are known to the skilled artisan.

The genes listed in Table 2 are merely for purposes of illustration, and are not in any way intended to mean that any one, combination or all genes must be detected, measured or analyzed, or that a minimum number of genes must be detected, measured or analyzed. Thus, additional genes not listed in Table 2, or expression products (proteins) encoded by such genes, can be detected, measured or analyzed, in accordance with the invention. For example, expression of additional protein coding genes, not listed in Table 2, whose position is altered as a consequence of a genomic sequence rearrangement, is potentially altered. Accordingly, in view of the guidance herein, any somatic chromosomal sequence rearrangement of a species conserved non-coding genomic sequence region, and expression of any coding gene whose position is altered relative to a chromosomal sequence rearrangement, is relevant for detection, measurement or analysis according to the methods, systems, databases, kits and arrays of the invention.

Accordingly, in another embodiment, altered expression of gene coding sequences, whose position is altered due to chromosomal sequence rearrangement, can be measured, detected or analyzed in order to predict the risk of or the presence or absence of a tumor or cancer. Altered expression of such genes (e.g., Table 2), relative to a normal comparison sample, can be used in accordance with the methods, systems, databases, kits and arrays of the invention.

Somatic chromosomal sequence rearrangements and/or gene expression products can be detected, measured or analyzed, as a combination of chromosomal sequence rearrangements, or a combination of gene expression products, particularly a plurality of somatic chromosomal sequence rearrangements and/or gene expression products. Accordingly, the invention includes detection, measurement or analysis of such a combination of somatic chromosomal sequence rearrangements and/or gene expression products.

As set forth herein, a somatic chromosomal sequence rearrangement correlates with an increased risk or presence of a tumor or cancer. Accordingly, absence of one or more somatic chromosomal sequence rearrangements correlates with a decreased risk of or absence of a tumor or cancer. A positive or negative result therefore indicates increased risk of or the presence or a decreased risk or absence of a tumor or cancer. As such, identification of a corresponding non-rearranged somatic chromosomal sequence is applicable for identifying low or no risk, or the absence of a tumor or cancer, in accordance with the invention.

The presence of a somatic chromosomal sequence rearrangement may be determined by sequencing the area of interest, or a nucleic acid derived therefrom, or analysis of a gene expression product, such as a polypeptide or protein. Additionally, the absence of a somatic chromosomal sequence rearrangement may be determined by sequencing the area of interest, or a nucleic acid derived therefrom, where presence of non-rearranged sequence indicates the absence of a somatic chromosomal sequence rearrangement.

Suitable nucleic acid samples for screening include genomic nucleic acid, such as genomic DNA. Suitable nucleic acid samples for screening also include nucleic acids derived from a genomic sequence, such as nucleic acid amplified from genomic nucleic acid (DNA), which can be referred to as a genomic nucleic acid amplification or synthesis product (e.g., amplified genomic nucleic acid). Such a nucleic acid derived from a genomic sequence reflects the genomic sequence since the genomic sequence (ultimately) served as a template for the derived nucleic acid. Accordingly, such nucleic acids derived from a genomic nucleic acid sequence are suitable for detecting, measuring or analyzing a somatic chromosomal sequence rearrangement since the sequence product would indicate the presence of the somatic chromosomal sequence rearrangement, if present, or indicate the absence of the somatic chromosomal sequence rearrangement.

A biological sample can be processed or manipulated in order to obtain genomic nucleic acid, and detect the presence of, or measure or analyze somatic chromosomal sequence rearrangements, or gene expression or expression product amounts or levels or function. Typically, a biological sample is processed to isolate a nucleic acid (e.g., total, genomic, or mRNA) or a gene expression product (e.g., a protein or fragment) that directly or indirectly is capable of indicating the presence or absence of somatic chromosomal sequence rearrangements, or an amount of a gene coding sequence expression product.

Biological samples include any sample capable of having a biological material, such as genomic nucleic acid or nucleic acid derived from genomic nucleic acid. Biological material includes cellular or genomic material, and cells. Biological samples therefore include a biological material or fluid or any material that includes genomic nucleic acid, such as genomic DNA, RNA or polypeptide (protein) suitable for detection, measurement or analysis of somatic chromosomal sequence rearrangements, or a gene whose expression is altered due to a somatic chromosomal sequence rearrangement (e.g., as set forth in Table 1). A biological sample therefore need only be suitable for detecting, measuring or analyzing somatic chromosomal sequence rearrangements or expression of one or more genes that correlate with a tumor or cancer prognosis, monitoring, or predictive outcome or treatment regime. Typically, biological samples include a cell, tissue or organ sample, such as a biopsy, or a sample from, blood, blood cells, serum, plasma, bone marrow, mucus, saliva, feces, cerebrospinal fluid, or urine.

Somatic chromosomal sequence rearrangements (and non-rearranged sequences) may be detected, measured or analyzed by sequence analysis of genomic nucleic acid (or a nucleic acid, such as a DNA derived therefrom), for example, genomic nucleic acid from a sample, such as a biological sample or material from a subject. Identification or rearranged or non-rearranged somatic chromosomal sequences can be performed by sequence analysis of the area of interest. In general, nucleic acid in a sample can be sequenced or detected by any suitable method or technique of sequence analysis or detection of a somatic chromosomal sequence rearrangement. For example, genomic sequence rearrangements can be detected, measured or analyzed by nucleic acid (genomic) sequencing, such as whole gene heteroduplex analysis, which has high levels of sensitivity.

“Sequence analysis” as used herein refers to determining a nucleotide sequence, e.g., that of a nucleic acid sequence, such as a genomic or other nucleic acid sequence (e.g., a genomic DNA, RNA or cDNA) or a product derived from a sequence, such as an amplification or synthesis product derived from a genomic sequence. The entire sequence or a partial sequence of a nucleotide sequence can be determined, and the determined nucleotide sequence can be referred to as a “read” or “sequence read.” In one embodiment, nucleic acids such as genomic sequences are analyzed directly without amplification (e.g., using single-molecule sequencing methodology). In other embodiments, nucleic acid sequences are amplified one or more times (e.g., 1-5, 5-10, 10-20, 10-30, 25-50 cycles) and the amplification product may be analyzed (e.g., using sequencing by ligation or pyrosequencing methodology). Any suitable sequencing method can be utilized to detect, measure or analyze the presence or absence of chromosomal sequence rearrangements, or detection of expression or an amount of a gene coding sequence, or an amplified or synthesized product generated from the foregoing.

Various sequencing techniques are known to one of skill in the art. One example of sequencing is whole genome sequencing. Examples of whole genome sequencing methods include, but are not limited to, nanopore-based sequencing methods, sequencing by synthesis and sequencing by ligation, as described further below.

Additional examples include primer extension methods (e.g., iPLEX; Sequenom, Inc.), microsequencing methods (e.g., a modification of primer extension methodology), ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), direct DNA sequencing, restriction fragment length polymorphism (RFLP analysis), allele specific oligonucleotide (ASO) analysis, pyrosequencing analysis, acycloprime analysis, GeneChip microarrays, Dynamic allele-specific hybridization (DASH), genetic bit analysis (GBA), Multiplex minisequencing, SNaPshot, Microarray miniseq, arrayed primer extension (APEX), microarray sequence determination methods (e.g., microarray primer extension), Microarray ligation, Ligase chain reaction (LCR), single strand conformational polymorphism analysis (SSCP), denaturing gradient gel electrophoresis (DGGE), heteroduplex analysis, and mismatch cleavage detection.

Pyrosequencing is a nucleic acid sequencing method based on sequencing by synthesis, which relies on detection of a pyrophosphate released on nucleotide incorporation. Pyrosequencing monitors DNA synthesis in real time using a luminometric detection system. Generally, sequencing involves synthesizing, one nucleotide at a time, a DNA strand complimentary to the strand whose sequence is being sought. Nucleic acids may be immobilized to a solid support, hybridized with a primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination. The amount of light generated is proportional to the number of bases added, and the sequence downstream of the sequencing primer is determined. Pyrosequencing has been used to analyze genetic polymorphisms (Nordstrom et al., Biotechnol. Appl. Biochem., 31:107 (2000); Ahmadian et al., Anal. Biochem., 280:103 (2000)). An exemplary system for pyrosequencing methodology is described in Nakano et al. (Journal of Biotechnology 102:117 (2003)).

Sequencing by ligation is a nucleic acid sequencing method that relies on sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for additional rounds of ligation.

Exemplary single-molecule sequencing methods are based on the principal of sequencing by synthesis, and utilize single-pair Fluorescence Resonance Energy Transfer (single pair FRET) as a mechanism by which photons are emitted after successful nucleotide incorporation. The emitted photons can be detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes (e.g., polymethine cyanine dyes Cy3 and Cy5), through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair” in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully. Examples of single-molecule sequencing systems are described in U.S. Pat. No. 7,169,314; and Braslaysky et al. (Proc. Natl. Acad. Sci. USA 100:3960 (2003)).

As disclosed herein, nucleotide sequencing may be by solid phase single nucleotide sequencing methods and processes. Solid phase single nucleotide sequencing methods involve contacting nucleic acid and solid support under conditions in which a single molecule of sample nucleic acid hybridizes to a single molecule of a solid support. Such conditions can include providing solid support molecules and a single molecule of target nucleic acid in a micro-reactor. Such conditions also can include providing a mixture in which the nucleic acid molecule can hybridize to solid phase nucleic acid on the solid support.

Sequencing detection methods also include contacting a nucleic acid for sequencing (e.g., genomic sequence) with sequence-specific detectors, under conditions in which the detectors specifically hybridize to the sequence (e.g., a rearranged or non-rearranged genomic sequence site, or a sequence derived therefrom). A signal from the detector indicates that the genomic sequence (e.g., a rearranged or non-rearranged genomic sequence site) is present. In certain methods, the detectors hybridized to the nucleic acid sequence are disassociated from the nucleic acid (e.g., sequentially dissociated) when the detectors interfere with a nanopore structure as the nucleic acid passes through a pore, and the detectors disassociated from the sequence are detected. In certain methods, a detector disassociated from a nucleic acid emits a detectable signal, and the detector hybridized to the nucleic acid emits a different detectable signal or no detectable signal thereby distinguishing one from the other.

Primer extension polymorphism detection methods, also referred to as “microsequencing” methods, typically are carried out by hybridizing a complementary oligonucleotide to a nucleic acid carrying the site of interest (e.g., the predicted location of the rearranged sequence site). In these methods, the oligonucleotide typically hybridizes adjacent to the site. The term “adjacent” used in reference to “microsequencing” methods refers to the 3′ end of the extension oligonucleotide being at least 1 nucleotide from the 5′ end of the site of interest, or more (e.g., 2-5, 5-10, 10-25, 25-50, 50-100, 100-500, or more) nucleotides from the 5′ end of the site of interest in the nucleic acid when the extension oligonucleotide is hybridized to the nucleic acid. The oligonucleotide is then extended by one or more nucleotides (e.g., labeled dideoxyribonucleotides), and the number and/or type of nucleotides that are added to the extension oligonucleotide determine whether the site of interest (e.g., the rearranged sequence site) is present. A labeled nucleotide is incorporated or linked to the primer only when the dideoxyribonucleotides matches the nucleotide at the sequence being detected. Thus, the identity of nucleotide(s) at the site of interest can be revealed based on the detection label attached to the incorporated dideoxyribonucleotides (e.g., Syvanen et al., Genomics, 8:684 (1990); Shumaker et al., Hum. Mutat., 7:346 (1996); and Chen et al., Genome Res., 10:549 (2000)).

Exemplary oligonucleotide extension methods are described, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; and 6,210,891. The extension products can be detected in any manner, such as by fluorescence methods (see, e.g., Chen and Kwok, Nucleic Acids Res. 25:347 (1997) and Chen et al., Proc. Natl. Acad. Sci. USA 94:10756 (1997)) mass spectrometric methods (e.g., MALDI-TOF mass spectrometry) and other methods. Exemplary oligonucleotide extension methods using mass spectrometry are described, for example, in U.S. Pat. Nos. 5,547,835; 5,605,798; 5,691,141; 5,849,542; 5,869,242; 5,928,906; 6,043,031; 6,194,144; and 6,258,538.

Microsequencing detection methods can incorporate an amplification process that precedes the extension step. The amplification process typically amplifies a region from a nucleic acid that includes the site of interest (e.g., the predicted location of the rearranged sequence site) Amplification can be carried out utilizing methods described herein, or for example using a pair of oligonucleotide primers in a polymerase chain reaction (PCR), in which one oligonucleotide primer typically is complementary to a region 3′ of the site of interest (e.g., the predicted location of the rearranged sequence site) and the other typically is complementary to a region 5′ of the polymorphism. Such methods are disclosed in U.S. Pat. Nos. 4,683,195; 4,683,202, 4,965,188; 5,656,493; 5,998,143; 6,140,054; WO 01/27327; and WO 01/27329, for example.

In certain sequence analysis methods, reads may be used to construct a longer nucleotide sequence, for example, by identifying overlapping sequences in different reads and by using identification sequences in the reads. Such sequence analysis methods and software for constructing larger sequences from reads are known to the person of ordinary skill (e.g., Venter et al., Science 291:1304 (2001)). Specific reads, partial nucleotide sequence constructs, and full nucleotide sequence constructs may be compared between nucleotide sequences within a nucleic acid (i.e., internal comparison) or may be compared with a reference sequence (i.e., reference comparison) in certain embodiments. A reference comparison can be performed when a reference nucleotide sequence is known and the objective is to determine whether a given nucleic acid sequence contains a nucleotide sequence of interest (e.g., rearranged sequence).

Sequence analysis can be facilitated by the use of sequence analysis instruments and components. A sequence analysis instrument or component includes an apparatus, and optionally one or more components used in conjunction with such apparatus, that can be used to determine a nucleotide sequence. Examples of sequencing instruments include, without limitation, the 454 platform (Roche) (Margulies et al., Nature 437:376 (2005)), Illumina Genomic Analyzer (or Solexa platform) or SOLID System (Applied Biosystems) or the Helicos True Single Molecule DNA sequencing technology (Harris et al., Science 320:106 (2008)), the single molecule, real-time (SMRT) technology (Pacific Biosciences), and nanopore sequencing. Such systems allow sequencing of many nucleic acid molecules at high orders of multiplexing in a parallel manner. Each of these instruments allows sequencing of clonally expanded or non-amplified single molecules of nucleic acid fragments.

In addition to sequencing methods, rearranged or non-rearranged somatic chromosomal sequences can be detected, analyzed or measured by nucleic acid probes (e.g., sequence-specific oligonucleotides) or other analytes that specially bind to the rearranged or non-rearranged somatic chromosomal sequences, or sequences (e.g., primers) that bind to sequences that flank the rearranged or non-rearranged somatic chromosomal sequence. As used herein “detecting,” “measuring” or “analyzing,” in the context of somatic chromosomal sequence rearrangement, a non-rearrangement or a gene refers to in solution, in solid phase, in vitro, in vivo or ex vivo methodology. Accordingly, detection, measurement or analysis includes in solution, in solid phase, in situ, in vitro, ex vivo, in a cell, such as a sample that includes cells in vivo, in vitro, in primary cell isolates, passaged cells, cultured cells, or cells ex vivo. Thus, contact includes conditions allowing the analyte to bind to another entity indicative of somatic chromosomal sequence rearrangements, non-rearrangements or a gene product, optionally including expression amounts and levels.

The term “bind,” or “binding,” means a physical interaction at the molecular level (directly or indirectly). Typically, binding is that which is specific or selective for a target, i.e., is statistically significantly higher than the background or control binding for the assay. The term “specifically binds” refers to the ability to preferentially or selectively bind to a target, for example, an analyte such as a polynucleotide, primer, probe, or antibody that binds to (or hybridizes with) a rearranged or non-rearranged somatic chromosomal sequence, or gene expression product. Specific and selective binding can be distinguished from non-specific binding using assays known in the art (e.g., for nucleic acid detection, polymerase chain reaction, DNA transcription, northern and southern blotting, etc., and or protein detection, immunoprecipitation, ELISA, flow cytometry, and Western blotting).

Compositions and methods of the invention may be contacted or provided in vitro, ex vivo or in vivo. The term “contact” and grammatical variations thereof means conditions allowing a physical interaction (direct or indirect) between two or more entities (e.g., an analyte and nucleic acid or expression product). In one example, contact means interaction (e.g., binding) of an analyte (e.g., polynucleotide, probe, primer, antibody or fragment, etc.) and genomic nucleic acid, such as that present in biological sample or material, or a cellular or other material derived from a biological sample.

Analytes according to the invention therefore include nucleic acid sequences. As used herein, the terms “nucleic acid” and “polynucleotide” and the like refer to at least two or more ribo- or deoxy-ribonucleic acid bases (nucleotides) that are linked through a phosphoester bond or equivalent covalent bond. Nucleic acids include polynucleotides and polynucleosides. Nucleic acids include single, double or triplex stranded, circular or linear, molecules. Nucleic acids include sense and anti-sense sequences, for example, sense and anti-sense sequences that bind to all or a portion of a chromosome sequence of interest, such as a rearranged sequence. Exemplary nucleic acids include but are not limited to: genomic nucleic acid, total RNA, mRNA, DNA, cDNA, naturally occurring and non-naturally occurring nucleic acid, e.g., synthetic or amplified nucleic acid.

Nucleic acids, such as genomic sequence rearrangements and synteny blocks can be of various lengths. Nucleic acid lengths typically range from about 10 nucleotides to 200 Mb, or any numerical value or range within or encompassing such lengths, e.g., 10 nucleotides to 10 Mb, 100 nucleotides to 5 Mb or less, 1,000 nucleotides to about 1 Mb, 5,000 nucleotides to about 500,000 nucleotides, 10,000 nucleotides to about 250,000 nucleotides, 25,000 nucleotides to about 100,000 nucleotides, or any numerical value or range or value within or encompassing such lengths. Nucleic acids can also be shorter, for example, 25,000, 10,000, or 5000 nucleotides or less, such as 500-1000 nucleotides, 100 to about 500 nucleotides, or from about 10 to 25, to 50, 50 to 100, 100 to 250, or about 250 to 500 nucleotides in length, or any numerical value or range or value within or encompassing such lengths. In particular aspects, a nucleic acid sequence has a length from about 10-20, 20-30, 30-50, 50-100, 100-150, 150-200, 200-250, 250-300, 300-400, 400-500, 500-1000, 1000-2000, 2,000-5,000, 5,000-10,000, 10,000-25,000, 25,000-50,000, 50,000-100,000, 100,000-250,000, 250,000-500,000, 500,000-1,000,000, 1,000,000-5,000,000, 5,000,000-10,000,000, 10,000,000-25,000,000, 25,000,000-50,000,000, 50,000,000-100,000,000, 100,000,000-200,000,000 nucleotides, or any numerical value or range within or encompassing such lengths.

Shorter polynucleotides are commonly referred to as “oligonucleotides” or “probes” or “primers” of single- or double-stranded DNA or RNA, or hybrids thereof, typically a length from about 8-20, 20-30, 30-50, 50-100, 100-200 nucleotides. Typically, they are single-stranded, but they can also be double-stranded having two complementary strands which can be separated by denaturation. Such shorter polynucleotides can be labeled with detectable markers or modified using conventional manners for various molecular biological applications.

Nucleic acids include, for example, polynucleotides and oligonucleotides (primers and probes) that hybridize to rearranged (such as those set forth herein) or non-rearranged somatic chromosomal sequences (or a transcript, RNA or cDNA thereof), for example. Such hybridizing nucleic acids allow detection of a target rearranged or non-rearranged somatic chromosomal sequence, or a complementary sequence, or a sequence derived therefrom, and can be used in accordance with the invention for screening, predicting or determining the risk of a tumor or cancer in a subject, as well as in the systems, organizational constructs, kits and arrays of the invention.

In order to detect, analyze or measure a rearranged or non-rearranged somatic chromosomal sequence, or detect, analyze or measure expression of a protein coding gene, a nucleic acid can “hybridize” to all or a portion of the rearranged or non-rearranged somatic chromosomal sequence, or complementary sequence, or sequence derived therefrom, or to a coding gene transcript or cDNA derived therefrom. Sequences “sufficiently complementary” allow stable hybridization of a nucleic acid sequence to a target sequence (such as a rearranged or non-rearranged somatic chromosomal sequence) and therefore detection even if the two sequences are not completely complementary. Detection may either be direct (i.e., resulting from a probe hybridizing directly to a sequence) or indirect (i.e., resulting from a probe hybridizing to an intermediate molecular structure that links the probe to the target sequence).

For example, sequence rearrangement specific probes (specific for binding to the rearranged sequence) can be used to specifically hybridize to a genomic sequence. The genomic nucleic acid (or nucleic acid derived therefrom) and the probe can be contacted with each other under conditions sufficiently stringent such that the rearranged sequence can be distinguished from the non-rearranged sequence based on the presence or absence of hybridization. The probe can be labeled to provide a detection signal.

Alternatively, sequence rearrangement specific probes (specific for binding to the rearranged sequence), or primer pairs adjacent to or flanking the sequence (the predicted location of the rearranged sequence) can be used as an amplification primer in a sequence-specific PCR. Again, the presence or absence of an amplified product of an expected length would indicate the presence or absence of a particular sequence rearrangement.

Hybridizing sequences will generally be more than about 50% complementary to all or a portion of a target sequence, such as a genomic sequence, a complementary sequence or a sequence derived from a genomic sequence. Typically, hybridizing sequences are 60%, 70%, 80%, 85%, 90%, or 95% complementary, or more to all or a portion of any of a genomic sequence target, or a sequence complementary to all or a portion of a genomic sequence. The hybridization region between hybridizing sequences typically is at least about 5-10, 10-15 nucleotides, 15-20 nucleotides, 20-30 nucleotides, 30-50 nucleotides, 50-75 nucleotides, 75-100 nucleotides, 100-200 nucleotides, 300-400 nucleotides, 400-500 nucleotides or more, or any numerical value or range within or encompassing such lengths.

The term “complementary” or “antisense” refers to a polynucleotide or peptide nucleic acid (PNA) capable of binding to all or a portion of a specific nucleic acid sequence (e.g., DNA or RNA sequence), such as a genomic sequence region of interest. Antisense includes single, double, triple or greater stranded RNA and DNA polynucleotides and peptide nucleic acids (PNAs) that bind RNA transcript or DNA. For example, a single stranded nucleic acid can target a genomic sequence of interest, such as a rearranged or non-rearranged somatic chromosomal sequence. Antisense/Sense molecules are typically 100% complementary to the sense/anti-sense strand but can be “partially” complementary, in which only some of the nucleotides bind to the sense/anti-sense molecule (less than 100% complementary, e.g., 95%, 90%, 80%, 70% and sometimes less), or any numerical value or range within or encompassing such percent values.

Polynucleotides useful as primers and probes in accordance with the invention typically include a portion/fragment of a genomic sequence (sense or anti-sense) suitable for use as a hybridization probe or primer for the detection, measurement or analysis of a genomic nucleic acid (or portion/fragment thereof) in a given sample (e.g., a sample comprising genomic nucleic acid), such as a rearranged or non-rearranged somatic chromosomal sequence. Typically, primers are oppositely oriented, (i.e., one primer positioned 5′, and a second primer positioned 3′) such that they can hybridize to and amplify the genomic nucleic acid sequence (e.g., via PCR), or a sequence derived from a genomic nucleic acid (e.g., a cDNA or RNA). Accordingly, in another embodiment, measuring includes hybridization of a primer pair (oppositely oriented) and subsequent amplification of a genomic sequence or a DNA/RNA derived from the genomic sequence, such as a rearranged or non-rearranged somatic chromosomal sequence.

Accordingly, in various embodiments, polynucleotides and oligonucleotides (primers and probes) for hybridization include (e.g., contact) an oligo- or poly-nucleotide probe to a genomic sequence, complementary sequence or a sequence derived from a genomic sequence (e.g., that specifically binds to a sequence rearrangement, such as a probe or primer), or to a protein coding gene sequence. In a particular embodiment, polynucleotides and oligonucleotides (primers and probes) for hybridization include (e.g., contact) an oligo- or poly-nucleotide probe that binds to a nucleic acid which allows detection of a genomic sequence, complementary sequence or a sequence derived from a genomic sequence (detection of a rearranged sequence or a non-rearranged sequence), or a protein coding gene sequence. Such sequences include fragments sufficient for detection or hybridization, and sequences that are 50%, 60%, 70%, 80%, 85%, 90%, or 95% identical to all or a portion of any sequence of a rearranged or non-rearranged somatic chromosomal sequence rearrangement as set forth herein, or gene coding sequence as set forth herein (e.g., Table 2).

The term “identity” and “homology” and grammatical variations thereof mean that two or more referenced entities are the same. Thus, where two sequences are identical, they have the same amino acid sequence, or are 100% identical or homologous. “Areas, regions or domains of identity” mean that a portion of two or more referenced entities are the same. Thus, where two sequences are identical or homologous over one or more sequence regions, they share identity in these regions. The term “complementary,” when used in reference to a nucleic acid sequence means the referenced regions are 100% complementary, i.e., exhibit 100% base pairing with no mismatches. Of course, reference to a sequence that is 90% complementary, means 90% base pairing with 10% sequence mismatches.

The degree of “identity” and “homology” can be determined by comparing each position in the sequences. A degree of identity or homology is a function of the number of identical or matching positions (e.g., matching nucleotides or amino acid residues) at positions shared by the sequences. Specific examples of “identity” and “homology” include a plurality of residues of the sequences. A sequence can have 50%, 60%, 70%, 75%, 80%, 85%, 90%, 95%, 96%, 97%, 98%, 99%, or more identity or homology to a reference sequence, to all or a portion of any of a genomic sequence, or a sequence derived from a genomic sequence. As used herein, a given percentage of identity or homology between sequences denotes the degree of sequence identity in optimally aligned sequences.

The extent of identity between two sequences can be ascertained using a computer program and mathematical algorithm. Such algorithms that calculate percent sequence identity (homology) generally account for sequence gaps and mismatches over the comparison region. For example, a BLAST (e.g., BLAST 2.0) search algorithm (see, e.g., Altschul et al., J. Mol. Biol. 215:403 (1990), publicly available through the National Center for Biotechnology Information, NCBI) has exemplary search parameters as follows: Mismatch-2; gap open 5; gap extension 2. The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance.

Hybridization between complementary regions of two strands of nucleic acid to form a duplex molecule will vary depending upon the nature of the hybridization method and the composition and length of the hybridizing nucleic acid sequences. Generally, temperature of hybridization and the ionic strength (such as the Na+ concentration) of the hybridization buffer will determine the stringency of hybridization (hybridization conditions for attaining particular degrees of stringency are discussed in Sambrook et al., (1989) Molecular Cloning, second edition, Cold Spring Harbor Laboratory, Plainview, N.Y.).

Exemplary non-limiting exemplary hybridization conditions are as follows:

Very High Stringency (Detects Sequences that Share 90% Identity)—Hybridization: 5×SSC at 65° C. for 16 hours, Wash twice in 2×SSC at room temperature (RT) for 15 minutes each, Wash twice in 0.5×SSC at 65° C. for 20 minutes each.
High Stringency (Detects Sequences that Share 80% Identity or Greater)—Hybridization: 5-6×SSC at 65° C.-70° C. for 16-20 hours, Wash twice in 2×SSC at RT for 5-20 minutes each, Wash twice: 1×SSC at 55° C.-70° C. for 30 minutes each.
Low Stringency (Detects Sequences that Share Greater than 50% Identity)—Hybridization: 6×SSC at room temp. to 55° C. for 16-20 hours, Wash at least twice in 2-3×SSC at room temp. to 55° C. for 20-30 minutes each.

In addition, gene product expression, which may be altered as a consequence of rearranged somatic chromosomal sequences may be measured and/or analyzed by any of a variety of methods known to one of skill in the art, such as with antibodies or activity/functional assays. Accordingly, detection, measuring and analysis of rearranged or non-rearranged somatic chromosomal sequences of gene coding sequences capable of encoding a protein can be determined by a variety of methods using various analytes.

As disclosed herein, gene expression can be measured and/or analyzed by detection of an expression product. As used herein, the term “expression product” is an amino acid sequence, protein, polypeptide, or peptide encoded by a gene or an exon. In particular, an expression product, for example, is encoded by all or a part of a gene set forth in Table 2. Invention methods, kits and arrays include detection, measurement or analysis of expression products encoded by one or more genes as set forth, for example, in Table 2.

Gene product expression (e.g., nucleic acid transcription) include detection, measurement or analysis of a transcript or corresponding cDNA. Accordingly, non-limiting exemplary methods of measuring gene product expression (e.g., nucleic acid transcription) include detection or analysis of a gene transcript. Methods for transcript detection, measurement and analysis include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other nucleic acid hybridization platform. For measuring RNA expression, methods include, but are not limited to: extraction of cellular mRNA and Northern blotting using labeled probes that hybridize to transcripts of all or part of one or more of the gene coding sequences set forth herein; amplification of mRNA expressed from one or more of the gene coding sequences (e.g., Table 2) using specific primers, polymerase chain reaction (PCR), quantitative PCR (q-PCR), and reverse transcriptase-polymerase chain reaction (RT-PCR), followed by quantitative detection of the product; and extraction of total RNA from cells, which is then processed (e.g. reverse transcribed or amplified), labeled and used to probe cDNAs or oligonucleotides encoding all or part of the gene coding sequences; and in situ hybridization.

Gene product expression also include detection, measurement or analysis of a protein. Accordingly, analytes in accordance with the invention further include molecules that bind to amino acid sequence, protein, polypeptide, or peptide encoded by all or a part of a gene (e.g., a sequence set forth in any of Table 2). As used herein the terms “amino acid sequence,” “protein,” “polypeptide” and “peptide” are used interchangeably to refer to two or more amino acids, or “residues,” covalently linked by an amide bond or equivalent. Exemplary lengths of such amino acid sequences are from about 5 to 10, 10 to 20, 20 to 25, 25 to 50, 50 to 100, 100 to 150, 150 to 200, or 200 to 300, 400 to 500, 500 to 1000, or more amino acid residues in length.

Analytes according to the invention therefore include ligands, antibodies and subsequences thereof that bind to proteins or fragments (peptides, polypeptides, etc.) encoded by the gene coding sequences. The term “antibody” refers to a protein that binds to other molecules (antigens) via heavy and/or light chain variable domains, V_{H} and/or V_{L}, respectively. An “antibody” refers to a monoclonal or polyclonal immunoglobulin molecule, such as IgG, IgA, IgD, IgE, IgM, and any subclass thereof (e.g., IgG₁, IgG₂, IgG₃ or IgG₄). Antibodies include full-length antibodies that include two heavy and two light chain sequences. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

A “monoclonal” antibody refers to an antibody that is based upon, obtained from or derived from a single clone, including any eukaryotic, prokaryotic, or phage clone. A “monoclonal” antibody is therefore defined structurally, and not the method by which it is produced.

Antibodies include subsequences. Non-limiting representative antibody subsequences include but are not limited to Fab, Fab′, F(ab)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}-V_{H})₂ or (V_{H}-V_{L})₂), triabody (trivalent), tetrabody (tetravalent), minibody ((scFᵥ-C_{H}3)₂), bispecific single-chain Fv (Bis-scFv), IgGdeltaCH2, scFv-Fc, (scFv)₂-Fc, affibody, aptamer, avimer or nanobody, or other antigen binding subsequences of an intact immunoglobulin. Antibodies include those that bind to more than one epitope (e.g., bi-specific antibodies), or antibodies that can bind to one or more different antigens (e.g., bi- or multi-specific antibodies).

Methods of detecting and measuring gene expression products, including for quantitation, are known to those of skill in the art. Non-limiting examples of protein detection, measurement and analysis methods include Western blot, immunoblot, enzyme-linked immunosorbant assay (ELISA), radioimmunoassay (RIA), immunoprecipitation, surface plasmon resonance, chemiluminescence, absorption, emission, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS) and flow cytometry. Amounts of expression products encoded by genes also include functional assays, based upon a function of the protein, such as enzyme or catalytic function, DNA binding function, ligand or receptor binding, signal transduction, etc.

The term “bind,” or “binding,” when used in reference to an analyte means that the binding moiety interacts at the molecular level with all or a part of a nucleic acid sequence, in order to detect, measure, or analyze rearranged or non-rearranged somatic chromosomal sequences, or a gene expression product (e.g., protein). Specific binding is selective for the sequence or expression product. Thus, selective binding to a rearranged somatic chromosomal sequence means that the sequence is present. In addition, binding to a corresponding non-rearranged somatic chromosomal sequence means that the sequence in question has not been rearranged, and the somatic chromosomal sequence rearrangement is absent. Specific and selective binding can be distinguished from non-specific binding using assays known in the art (e.g., immunoprecipitation, ELISA, flow cytometry, immunohistochemistry, Western blotting, nucleic acid hybridization, etc.).

An analyte can be labeled or tagged in order to be detectable. Detectable labels, markers and tags include labels suitable for somatic chromosomal sequence or expression product detection, measurement, analysis and/or quantitation, and include any composition detectable by enzymatic, biochemical, spectroscopic, photochemical, immunochemical, isotopic, electrical, optical, chemical or other means. A detectable label can be attached (e.g., linked conjugated) to the analyte, or be within or be one or more atoms that comprise the analyte. As the structure of analytes can include one or more of carbon, hydrogen, nitrogen, oxygen, sulfur, phosphorous, etc., radioisotopes of any of carbon, hydrogen, nitrogen, oxygen, sulfur, phosphorous, etc., can be included within an analyte detectably labeled.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of C, N, O, H, S, Cu, Fe, Ga, Ti, Sr, Y, Tc, In, Pm, Gd, Sm, Ho, Lu, Re, At, Bi or Ac. In additional embodiments, a radioisotope can be one or more of ³H, ¹¹C, ¹⁴C, ¹³N, ¹⁸O, ¹⁵O, ³²P, ³³P, ³⁵S, ¹²⁵I or ¹³¹I.

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a colorimetric labels such as colloidal gold or colored glass or plastic (e.g., polystyrene, polypropylene, latex, etc.) beads; a fluorescent material or dye (e.g., umbelliferone, fluorescein, fluorescein isothiocyanate, rhodamine, dichlorotriazinylamine fluorescein, dansyl chloride, texas red, rhodamine); a luminescent material (e.g., luminol); or a bioluminescent material (e.g., green fluorescent protein, luciferase, luciferin, aequorin). A label can be any imaging agent that can be employed for gene expression or expression product detection, measurement, analysis and/or quantitation (e.g., for computed axial tomography (CAT or CT), fluoroscopy, single photon emission computed tomography (SPECT) imaging, optical imaging, positron emission tomography (PET), magnetic resonance imaging (MRI), gamma imaging).

A detectable label can also be linked or conjugated (e.g., covalently) to the analyte. In various embodiments a detectable label, such as a radionuclide or metal or metal oxide can be bound or conjugated to the analyte, either directly or indirectly. A linker or an intermediary functional group can be used to link an analyte to a detectable label.

An analyte (i.e., nucleic acid, protein, antibody or fragment thereof) can be either in a free state, in solution or in solid phase, such as immobilized on a substrate or a support (e.g., solid). Examples of substrates and supports include a multiwall plate, a chip, a bead or sphere, a tube or vial, a microarray or any other suitable substrate or support. For example, a nucleic acid, such as a probe or plurality of probes can be divided up and individual members presented in microtiter wells or used as probes in Fluorescence In-Situ Hybridization (FISH) Immobilization can be by passive adsorption (non-covalent binding) or covalent binding between the substrate or support and the analyte, or indirectly by attaching the analyte to a reagent which reagent is then attached to the substrate or support.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as eukaryote or mammalian cell, yeast or bacteria, in an animal or in a plant).

In various embodiments of the invention, genomic nucleic acid is amplified, for example, using short, medium or long range polymerase chain reaction (PCR). Amplification is useful for detecting (e.g., sequencing) small quantities of nucleic acid. Amplification is also useful where only small sample quantities are available. Primers can be used to amplify a selected region, which amplified regions can be relatively short, e.g., 20-100 base pairs, or longer, for example, over 100 or more (e.g., 100-1,000), 1,000 or more (e.g., 1,000-5,000), 5,000 or more (e.g., 5,000-10,000), 10,000 or more (e.g., 10,000-25,000), 25,000 or more (e.g., 25,000-50,000), 50,000 or more (e.g., 50,000-100,000), or 100,000 or more (e.g., 100,000 or more, 200,000 or more, 300,000 or more, 400,000 or more, or 500,000 or more, e.g., 100,000-1,000,000), or 1,000,000 or more (e.g., 1,000,000-10,000,000, 10,000,000-25,000,000, etc.) base pairs.

In certain embodiments, the entire genomic DNA from all sample cells is amplified to the same extent (“whole genome amplification,” or WGA), such that the sequence of genomic DNA (e.g. normal and abnormal parts of the genome) is maintained in the amplified product as compared to the original sample. The whole genome of a sample may be amplified according to this method prior to sequence analysis. This unbiased amplification provides a sequence profile for each sample, which profiles can be further used to detect, measure or analyze somatic genomic sequence rearrangements and correlation with a tumor or cancer.

In other embodiments, genomic nucleic acid may be selectively amplified, such that only a part of the whole genome, such as s particular sequence region, is amplified for sequence analysis. For example, if a particular genomic sequence rearrangement is known to occur in a particular genomic sequence region, it is possible to selectively amplify genomic regions associated with the particular genomic sequence rearrangement. These selectively amplified genomic sequence regions will provide the same information as to the presence or absence of genomic sequence rearrangements, but with enhanced sensitivity (e.g. capable of detecting genomic sequence rearrangements in smaller amounts of sample) and larger signal/noise ratio (since the proportion of the relevant genomic sequence has increased by amplification).

Many suitable amplification methods are applicable for use in accordance with the invention. A non-limiting example is polymerase chain reaction (PCR), which amplifies nucleic acid by repeated thermal denaturation, primer annealing and polymerase extension, thereby amplifying a single target nucleic acid sequence to greater quantities. PCR is typically used to amplify regions of DNA up to about 10,000 bases.

In particular aspects, the genomic nucleic acid is amplified by whole genome PCR, Lone Linker PCR, Interspersed Repetitive Sequence PCR, Linker Adapter PCR, Priming Authorizing Random Mismatches-PCR, single cell comparative genomic hybridization (SCOMP), degenerate oligonucleotide-primed PCR (DOP-PCR), Sequence Independent PCR, Primer-extension pre-amplification (PEP), improved PEP (I-PEP), Tagged PCR (T-PCR), tagged random hexamer amplification (TRHA); or using rolling circle amplification (RCA), multiple displacement amplification (MDA), or multiple strand displacement amplification (MSDA). The following methods for producing amplified sequences, which is useful for detecting, measuring or analyzing genomic sequences in accordance with the invention are merely exemplary, as additional methods are known to those of skill in the art (see, e.g., U.S. Pat. Nos. 6,107,023; 6,114,149; 6,124,120; 6,280,949; 6,365,375; and WO 04/111266)

Whole genome PCR amplifies either complete pools of DNA or unknown intervening sequences between specific primer binding sites. The amplification of complete pools of DNA, termed “known amplification” (or “general amplification” can be achieved by different means. The method is capable of unanimously amplifying nucleic acid fragments in the reaction mixture without preference for specific sequences. Primers used for whole genome PCR are totally degenerate (i.e., all nucleotides are termed N,N=A, T, G, C), partially degenerate (i.e., several nucleotides are termed N) or non-degenerate (i.e., all positions exhibit defined nucleotides).

Whole genome PCR involves fragmenting total genomic nucleic acid via shearing or enzymatic digestion with, for instance, a restriction enzyme, to an average size of 200-300 base pairs. The ends of the DNA are made blunt by incubation with Klenow fragment of DNA polymerase, and the fragments are ligated to catch linkers consisting of a 20 base pair DNA fragment. The linked DNA can be amplified by PCR™ using the catch oligomers as primers, and a DNA of interest can then be selected via binding to a specific protein or nucleic acid and recovered.

Lone Linker PCR employs asymmetrical linkers for the primers and produces fragments ranging from 100 bases to about 2 kb. The sequences of the catch linker oligonucleotides are used with the exception of a deleted 3 base pair sequence from the 3′-end of one strand. This “lone-linker” has both a non-palindromic protruding end and a blunt end, thus preventing multimerization of the catch linkers. Moreover, as the orientation of the linker was defined, a single primer is sufficient for amplification. After digestion with a four-base cutting enzyme, the lone linkers are ligated.

Interspersed Repetitive Sequence PCR uses non-degenerate primers that are based on repetitive sequences within the genome. This amplifies segments between suitable positioned repeats and has been used to create human chromosome- and region-specific libraries. IRS-PCR is also termed Alu element mediated-PCR (ALU-PCR), which uses primers based on the most conserved regions of the Alu repeat family and allows the amplification of fragments flanked by these sequences. A disadvantage of IRS-PCR™ is that abundant repetitive sequences like the Alu family are not uniformly distributed throughout the human genome, but preferentially found in certain areas (e.g., the light bands of human chromosomes). Thus, IRS-PCR™ results in a bias toward these regions and a lack of amplification of other, less represented areas. This technique is dependent on the knowledge of the presence of abundant repeat families in the genome of interest.

Linker Adapter PCR addresses limitations of IRS-PCR by using the linker adapter technique (LA-PCR). This technique amplifies unknown restricted DNA fragments with the assistance of ligated duplex oligonucleotides (linker adapters). DNA is commonly digested with a frequently cutting restriction enzyme, yielding fragments that are on average 500 bp in length. After ligation, PCR is performed using primers complementary to the sequence of the adapters. Temperature conditions are selected to enhance annealing specifically to the complementary DNA sequences, which leads to the amplification of unknown sequences situated between the adapters. Post-amplification, the fragments are cloned. There should be little sequence selection bias with LA-PCR™ except on the basis of distance between restriction sites. Methods of LA-PCR overcome the hurdles of regional bias and species dependence common to IRS-PCR.

Priming Authorizing Random Mismatches PCR is another whole genome PCR method using non-degenerate primers (PARM-PCR). This method uses specific primers and low stringency annealing conditions resulting in a random hybridization of primers leading to universal amplification. Annealing temperatures are reduced to 30° C. for the first two cycles and increased to 60° C. in subsequent cycles to specifically amplify the generated DNA fragments. This method has been used to universally amplify chromosomes for identification via fluorescent in situ hybridization (FISH).

The Single Cell Comparative Genomic Hybridization method allows the comprehensive analysis of the entire genome on a single cell level (SCOMP) (WO 00/17390). Genomic DNA from a single cell is fragmented with a four base restriction enzyme (e.g., MseI) producing fragments of predicted average length of 256 bp—based on the assumption that the four bases are evenly distributed. Ligation mediated PCR was utilized to amplify the digested restriction fragments. Briefly, primers are annealed to each other to create an adapter with two 5′ overhangs. The 5′ overhang resulting from the shorter oligo is complementary to the ends of the DNA fragments produced by MseI cleavage. The adapter was ligated to the digested fragments using T4 DNA ligase. Only the longer primer was ligated to the DNA fragments as the shorter primer did not have the 5′ phosphate necessary for ligation. Following ligation, the second primer was removed via denaturation, and the first primer remained ligated to the digested DNA fragments. The resulting 5′ overhangs were filled in by the addition of DNA polymerase. The resulting mixture was then amplified by PCR using the longer primer. Because this method relies on restriction digests to fragment genomic nucleic acid, typically very small and very long restriction fragments will not be effectively amplified, resulting in a biased amplification.

Alternative methods have been developed to overcome certain limitations associated with using non-degenerate primers for universal amplification, by using partially or totally degenerate primers.

Degenerate oligonucleotide-primed PCR (DOP-PCR), which has been applied to less than one nanogram of starting genomic nucleic acid, was developed using partially degenerate primers, thus providing a more general amplification technique. DOP-PCR is based on the principle of priming from short sequences specified by the 3′-end of partially degenerate oligonucleotides used during initial low annealing temperature cycles. As these short sequences occur frequently, amplification of target sequences proceeds at multiple loci simultaneously. As an example, non-specific primers showing complete, degeneration at positions 4, 5, 6, and 7 from the 3′ end were used. The three specific bases at the 3′ end are statistically expected to hybridize every 64 (43) bases, thus the last seven bases will match due to the partial degeneration of the primer. Amplification occurs in two stages, the first is at low temperature cycles, and in the second annealing is performed at a temperature restricting non-specific hybridization. The first cycles of amplification are conducted at a low annealing temperature (e.g., 30° C.), allowing sufficient priming to initiate DNA synthesis at frequent intervals along the template. The defined sequence at the 3′ end of the primer tends to separate initiation sites, thus increasing product size. As the PCR product molecules all contain a common specific 5′ sequence, in subsequent cycles the annealing temperature is raised for example, after the first eight cycles.

Another adaptation of the DOP-PCR method has been described that produces long products ranging from 0.5 to 7 kb in size, allowing amplification of long sequence targets in subsequent PCR (long DOP-PCR). This long DOP-PCR was reported to use 200 ng of genomic DNA. Subsequently, a method was described that generates long amplification products from less (e.g., picogram) quantities of genomic nucleic acid, termed long products from low DNA quantities DOP-PC (LL-DOP-PCR). This method achieves this by the 3-5′ exonuclease proofreading activity of DNA polymerase Pwo and an increased annealing and extension time during DOP-PCR, which are steps that generate longer products.

Sequence Independent PCR is an approach using degenerate primers, called sequence-independent DNA amplification (SIA). In contrast to DOP-PCR, SIA incorporates a nested DOP-primer system. As an example, the first primer consisted of a five base random 3′-segment and a specific 16 base segment at the 5′ end containing a restriction enzyme site. Stage one of PCR starts at 97° C. for denaturation, followed by cooling to 4° C., causing primers to anneal to multiple random sites, and then heating to 37° C. A T7 DNA polymerase is used. In the second low-temperature cycle, primers anneal to products of the first round, and the primer contains, at the 3′ end, 15 5′-end bases of primer A. Five cycles were performed with this primer at an intermediate annealing temperature of 42° C. An additional 33 cycles we performed at a specific annealing temperature of 56° C. Products of SIA ranged from 200 bp to 800 bp.

Primer-extension Pre-amplification (PEP) is a method that uses totally degenerate primers to achieve universal amplification of the genome. PEP uses a random mixture of 15-base fully degenerate oligonucleotides as primers—any one of the four possible bases could be present at each position. Theoretically, the primer is composed of a mixture of 4×10⁹ different oligonucleotide sequences, which leads to amplification of DNA sequences from randomly distributed sites. In each of the 50 cycles, the template is first denatured at 92° C., and subsequently, primers are allowed to anneal at a low temperature (37° C.), which is then continuously increased to 55° C. and held for another four minutes for polymerase extension.

An improved PEP (I-PEP) method was developed to enhance efficiency of PEP, primarily for the investigation of tumors from tissue sections used in routine pathology to reliably perform multiple microsatellite and sequencing studies with a single or few cells. I-PEP differs from PEP in cell lysis approaches, improved thermal cycle conditions, and the addition of a higher fidelity polymerase—cell lysis was performed in EL buffer, Taq polymerase is mixed with proofreading Pwo polymerase, and an additional elongation step at 68° C. for 30 seconds before the denaturation step at 94° C. was added. I-PEP was more efficient than PEP and DOP-PCR in amplification of DNA from one cell and five cells.

Tagged PCR (T-PCR) was developed to increase amplification efficiency of PEP in order to amplify efficiently from small quantities of nucleic acid with amplified sizes ranging from 400 bp to 1.6 kb. T-PCR is a two-step strategy, which uses, for the first few low-stringent cycles, a primer with a constant 17 base pair at the 5′ end and a tagged random primer containing 9 to 15 random bases at the 3′ end. In the first step, the tagged random primer is used to generate products with tagged primer sequences at both ends, which is achieved by using a low annealing temperature. The unincorporated primers are then removed and amplification is carried out with a second primer containing only the constant 5′ sequence of the first primer under high-stringency conditions to allow exponential amplification. This method requires removal of unincorporated degenerate primers, which also can cause loss of sample material. Loss of genomic sequence template during the purification steps could affect the coverage of T-PCR.

Tagged Random Hexamer Amplification (TRHA) was developed to address limitations of T-PCR, and uses a tagged random primer with shorter random bases. In TRHA, the first step is to produce a size distributed population of DNA molecules from a pNL1 plasmid, which can be done via a random synthesis reaction using Klenow fragment and random hexamer tagged with T7 primer at the 5′-end. Klenow-synthesized molecules (size range 28 bp-<23 kb) were then amplified with T7 primer. Examination of bias indicated that only 76% of the original DNA template was preferentially amplified and represented in the TRHA products.

Strand Displacement is an isothermal technique of rolling circle amplification for amplifying large circular DNA templates such as plasmid and bacteriophage DNA. Using 029 DNA polymerase, which synthesizes DNA strands 70 kb in length using random exonuclease-resistant hexamer primers, DNA was amplified in a 30° C. isothermal reaction. Secondary priming events occur on displaced product DNA strands, resulting in amplification via strand displacement. Two sets of primers are used. The right set of primers each have a portion complementary to nucleotide sequences flanking one side of a target nucleotide sequence, and primers in the left set of primers each have a portion complementary to nucleotide sequences flanking the other side of the target nucleotide sequence. The primers in the right set are complementary to one strand of the nucleic acid molecule containing the target nucleotide sequence, and the primers in the left set are complementary to the opposite strand. The 5′ end of primers in both sets is distal to the nucleic acid sequence of interest when the primers are hybridized to the flanking sequences in the nucleic acid molecule. Ideally, each member of each set has a portion complementary to a separate and non-overlapping nucleotide sequence flanking the target nucleotide sequence. Amplification proceeds by replication initiated at each primer and continuing through the target nucleic acid sequence. Once the nucleic acid strands elongated from the right set of primers reaches the region of the nucleic acid molecule to which the left set of primers hybridizes, and vice versa, another round of priming and replication commences, which allows multiple copies of a nested set of the target nucleic acid sequence to be synthesized.

Multiple Displacement Amplification is a technique, a random set of primers is used to prime a sample of genomic DNA, based upon the assumption that random primers equally prime over the entire genome, thus allowing representative amplification. By selecting a sufficiently large set of primers of random or partially random sequence, the primers in the set will be collectively, and randomly, complementary to nucleic acid sequences distributed throughout nucleic acids in the sample Amplification proceeds by replication with a highly possessive polymerase, φ29 DNA polymerase, initiating at each primer and continuing until spontaneous termination. Displacement of intervening primers during replication by the polymerase allows multiple overlapping copies of the entire genome to be synthesized. This technique is useful in studying specific loci, but random-primed amplification products typically are not equally representative of the starting material (e.g., the entire genome).

In embodiments in which nucleic acid is amplified, whatever amplification method is used, if a result is desired that reflects gene expression amounts or levels, a method is used that maintains or controls for the relative frequencies of the amplified nucleic acids to achieve quantitative amplification. Various methods of “quantitative” amplification are known to those skilled in the art. For example, quantitative PCR involves simultaneously co-amplifying a known quantity of a control sequence using the same primers. This provides an internal standard that may be used to calibrate the PCR reaction. Thus, primers and/or probes specific to the internal standard can be used for quantification of the amplified nucleic acid. Other suitable amplification methods include, but are not limited to polymerase chain reaction (PCR; Innis, et al., PCR Protocols. A Guide to Methods and Application. Academic Press, Inc. San Diego, (1990)), ligase chain reaction (LCR; Wu and Wallace, Genomics, 4:560; Landegren et al., Science, 241: 1077; and Barringer, et al., Gene, 89:117)), transcription amplification (Kwoh et al., Proc. Natl. Acad. Sci. USA, 86:1173), and self-sustained sequence replication (Guatelli et al., Proc. Nat. Acad. Sci. USA, 87:1874). Accordingly, gene expression levels may in general be measured or analyzed by detecting RNA, such as mRNA from cells (or cDNA thereof) and/or detecting gene expression products, such as a polypeptide or protein.

Genomic sequence rearrangements can be detected, measured or analyzed individually, or a plurality of such sequence rearrangements can be detected, measured or analyzed in cells of a subject (or a sample) in order to predict or determine the risk of, the presence of, or monitor development or progression of a tumor or cancer. Genomic sequence rearrangements and potentially affected genes whose expression may be altered as a consequence of such a rearrangement, may be analyzed in combination. Accordingly, a plurality of analytes (e.g., polynucleotides such as probes or primer pairs) can be used in accordance with the invention. Multiple polynucleotides (e.g., probes or primer pairs) can be used to detect, measure or analyze a plurality of genomic sequence rearrangements (e.g., any rearrangement of Table 1), corresponding non-rearrangements, or gene expression products (e.g., any genes of Table 2).

As used herein, the term “plurality” means 2 or more. As set forth herein, a plurality of somatic chromosomal sequence rearrangements can be detected, measured or analyzed. Thus, 2 or more rearrangements (e.g., Table 1) or genes coding sequences (e.g., Table 2) can be measured or analyzed in accordance with the invention. In particular embodiments, the number of somatic chromosomal sequence rearrangements and/or gene coding sequences detected, measured or analyzed is 2, 3, 4, 5, 6, 7, 8, 9, 10, 11, 12, 13, 14, 15, 16, 17, 18, 19, 20 or more (e.g., 21, 22, 23, 24, 25, etc.).

Likewise, a plurality of analytes (e.g., probes, primers, or antibodies) in the methods, systems, databases, kits and/or arrays can be used to detect a somatic chromosomal sequence rearrangement (e.g., Table 1), or non-rearrangement, or expression products (proteins) encoded by coding genes (e.g., Table 2). Thus, analytes (e.g., primers, probes or antibodies) in accordance with the invention can include those that detect somatic chromosomal sequence rearrangements (Table 1), non-rearrangements, or gene products (proteins) listed in Table 2.

Tumor or cancer prediction and/or identifying, monitoring, analysis, classifying, categorizing, scoring for risk or assessment according to one or more somatic chromosomal sequence rearrangements is based upon one or more somatic chromosomal sequence rearrangements, or the totality of the number and type of somatic chromosomal sequence rearrangements. A somatic chromosomal sequence rearrangement profile refers to a plurality of somatic chromosomal sequence rearrangements, or is a dataset of one or more somatic chromosomal sequence rearrangements, optionally compared to a respective normal cell, or optionally correlating with risk of or the presence of a tumor or cancer. The number and type of somatic chromosomal sequence rearrangements is considered to indicate the type, severity, progression or advancement of tumor or cancer, and can in turn be represented by a score.

Accordingly, a score can be based upon a chromosomal sequence rearrangement profile, or expression of a coding gene(s), or the totality of such information. The score can reflect a subject's probability or degree of risk of a tumor or cancer, the presence or absence of the tumor or cancer. The score can also reflect a class or stage (e.g., development, progression or worsening, or regression), which can indicate diagnosis, prognosis, clinical outcome or severity, or a treatment regime tailored for the tumor or cancer.

A risk score can be compared to a predefined or predetermined reference score. For example, a predefined or predetermined reference score can be set according to the type or number of somatic chromosomal sequence rearrangements (or altered gene coding sequence expression) that predict a tumor or cancer, or that reflect an increased risk of a tumor or cancer. A risk score greater than the predefined or predetermined risk score can reflect the presence or an increased risk of the tumor or cancer, and a risk score less than the predefined or predetermined risk score can reflect the absence or reduced risk of a tumor or cancer. The reference score can be set to a higher or lower threshold. Generally, to reduce or minimize the risk or probability of a false negative for a tumor or cancer, the user can select for a lower reference score.

In accordance with the invention, where a plurality of somatic chromosomal sequence rearrangements are detected, measured or analyzed, typically there will be a threshold (e.g., minimum) number or type of somatic chromosomal sequence rearrangements, or expression levels or amounts of coding genes, in order to predict or determine that the subject has or is at high risk, or does not have or is at low risk, of a tumor or cancer. Accordingly, a threshold number or type of somatic chromosomal sequence rearrangements can be set and, for example, be based upon the desire to minimize false negatives, or to increase the degree of confidence or accuracy of tumor or cancer prediction, monitoring, or data or information. Such a number can be only one, but may be greater, e.g., 2-5, 5-10, or more.

Subjects include animals, typically vertebrate or mammalian animals (mammals), such as humans, non human primates (apes, gibbons, chimpanzees, orangutans, macaques), domestic animals (dogs and cats), farm animals (horses, cows, goats, sheep, pigs) and experimental animal (mouse, rat, rabbit, guinea pig). In accordance with the invention, appropriate subjects include those having or at risk of having a metastatic or non-metastatic tumor, cancer, malignant or neoplastic cell, those undergoing as well as those who have undergone anti-proliferative (e.g., metastatic or non-metastatic tumor, cancer, malignancy or neoplasia) therapy, including subjects where the tumor is in remission.

Appropriate subjects also include those “at risk” of a tumor or cancer, whom typically have risk factors associated with development of hyperplasia (e.g., a tumor or cancer). At risk subjects include those that are candidates for and those that have undergone surgical resection, chemotherapy, immunotherapy, ionizing or chemical radiotherapy, or local or regional thermal (hyperthermia) therapy. The invention is therefore applicable to subjects at risk of a metastatic or non-metastatic tumor, cancer, malignancy, or neoplasia, for example, due to metastatic or non-metastatic tumor, cancer, malignancy or neoplasia reappearance or regrowth following a period of stability or remission.

Data or information based upon the presence or absence of somatic chromosomal sequence rearrangements, and any correlations with a tumor or cancer, may be represented by any form. The data or information may be presented as a physical representation (e.g., paper, such as a graph), computer (e.g., on a screen) or digital representation or as data stored in an electronic or computer-readable medium. Such data can be accessed by a user, for example, to input a query sample from a subject of one or more somatic chromosomal sequence rearrangements in order to perform a diagnosis or monitoring a tumor or cancer of the subject.

In accordance with the invention, further provided are systems, databases and organizational constructs. A “database” or “organizational construct” typically includes information. Information includes, but is not limited to, a correlation between one or more somatic chromosomal sequence rearrangements and the risk or probability, or the presence or diagnosis of tumor or cancer, or progression, clinical outcome, or treatment regime for a tumor or cancer, or sample analysis that indicates the presence or absence of one or more somatic chromosomal sequence rearrangements predictive of the risk or probability, or the presence or diagnosis of a tumor or cancer, or progression, clinical outcome, or treatment regime for a tumor or cancer. Invention systems, databases and organizational constructs can be operatively linked to a processor, such as a processor that includes a data entry module or a query module.

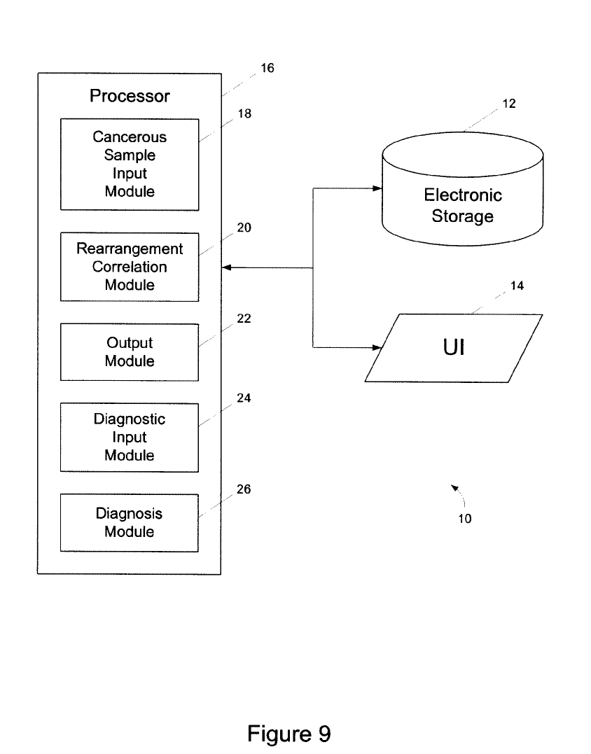

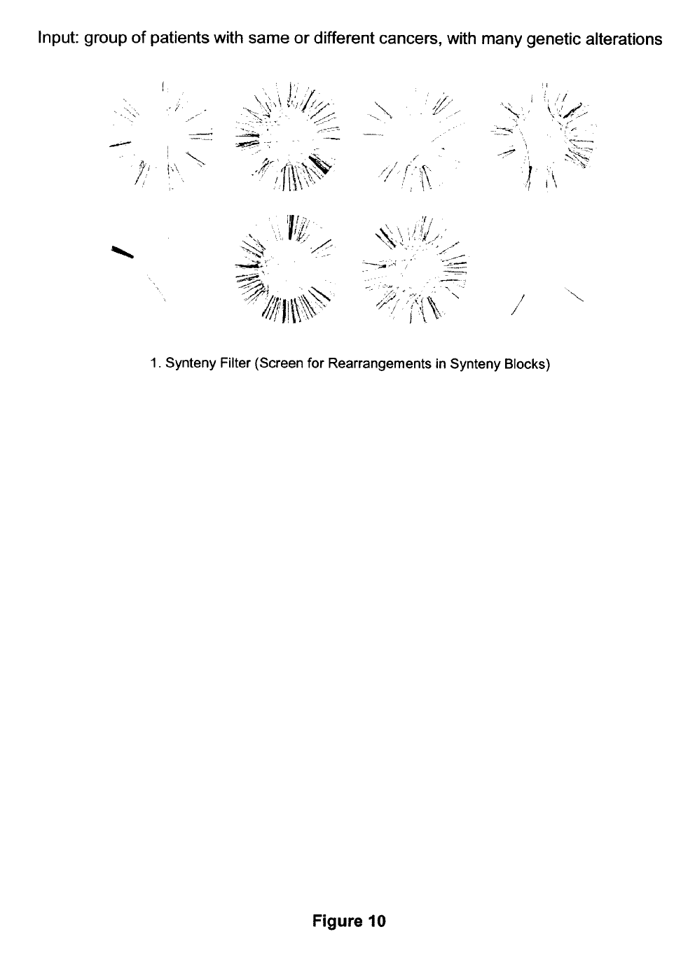

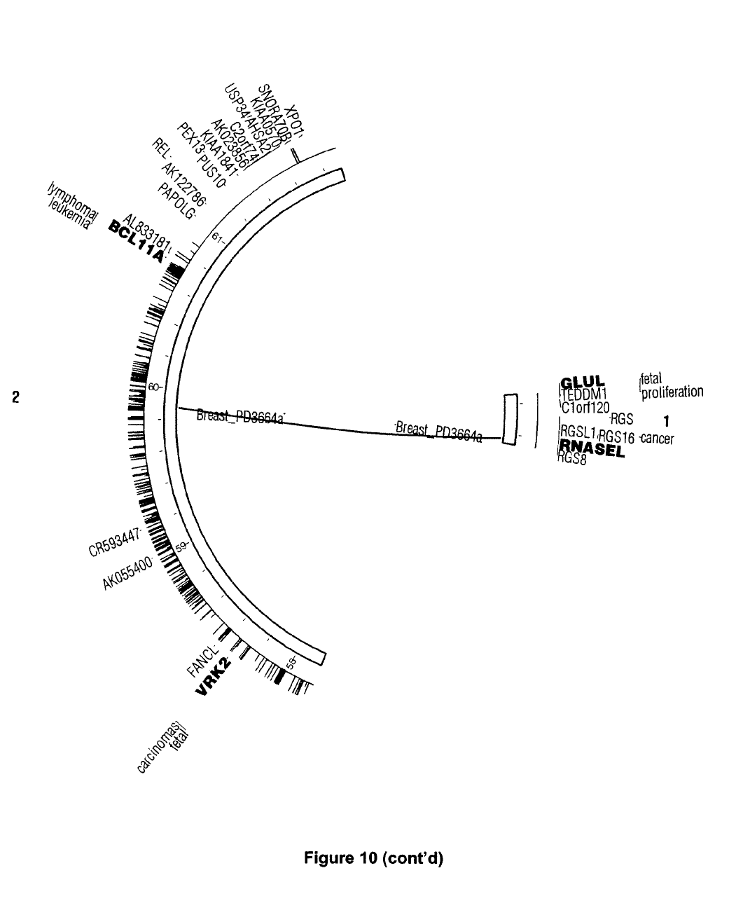

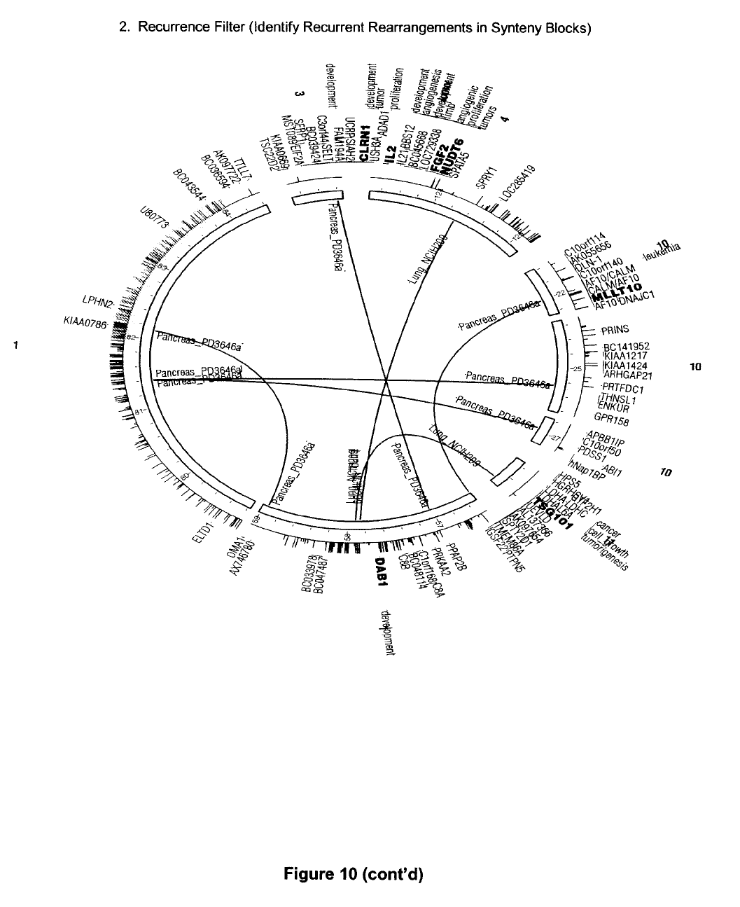

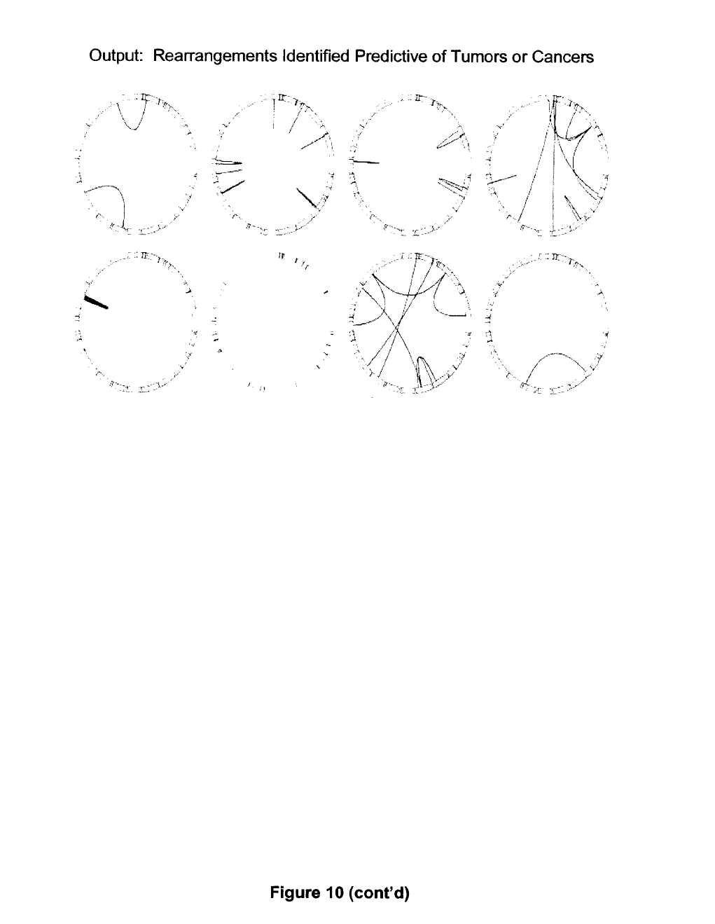

FIG. 9 illustrates an exemplary system 10 to correlate chromosomal sequence rearrangements and the risk or probability, or the presence or diagnosis of tumor or cancer, or progression, clinical outcome, or treatment regime for a tumor or cancer. The system 10 may be configured to implement the techniques related to identifying and/or leveraging relationships between chromosomal sequence rearrangements and the presence of a tumor or cancer, or an increased risk of a tumor or cancer. The system 10 may include one or more of electronic storage 12, a user interface 14, a processor 16, and/or other components.

Electronic storage 12 comprises electronic storage media that electronically stores information. The electronic storage media of electronic storage 12 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 12 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), network-based media (e.g., cloud storage), and/or other electronically readable storage media. Electronic storage 12 may include virtual storage resources, such as storage resources provided via a cloud and/or a virtual private network. Electronic storage 12 may store software algorithms, information determined by processor 16, information received via user interface 14, and/or other information that enables system 10 to function properly. Electronic storage 12 may be a separate component within system 10, or electronic storage 12 may be provided integrally with one or more other components of system 10 (e.g., processor 16).

User interface 14 is configured to provide an interface between system 10 and a user through which the user may provide information to and receive information from system 10. This enables data, results, and/or instructions and any other communicable items, collectively referred to as “information,” to be communicated between the user and one or more of electronic storage 12, processor 16, and/or other components of system 10. Examples of interface devices suitable for inclusion in user interface 14 include a keypad, buttons, switches, a keyboard, knobs, levers, a display screen, a touch screen, speakers, a microphone, an indicator light, an audible alarm, and a printer.

It is to be understood that other communication techniques, either hard-wired or wireless, are also contemplated by the present invention as user interface 14. For example, the invention contemplates that user interface 14 may be integrated with a removable storage interface provided by electronic storage 12. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 14 include, but are not limited to, an RS-232 port, RF link, an IR link, modem (telephone, cable or other). In short, any technique for communicating information with system 10 is contemplated by the present invention as user interface 14.

In some embodiments, system 10 may include a client/server architecture in which user interface 14 is presented to users by a client computing platform in communication with a server computing platform. The client computing platform may include one or more of a desktop computer, a laptop computer, a personal digital assistant, a tablet computing platform, a handheld computer, a Smartphone, mobile telephone, and/or other client computing platforms. The client computing platform may include one or more processors configured to execute a client application that interfaces with the server computing platform. The client application may be a dedicated client application configured specifically to perform the tasks and/or functions described herein. The client application may include a multi-purpose application (e.g., a web browser) configured to communicate with the server computing platform. Communication between the client computing platform and the server computing platform may accomplished via wired and/or wireless communication media. Communication may be accomplished via a network and/or dedicated communication lines.

Processor 16 is configured to provide information processing capabilities in system 10. As such, processor 16 may include one or more of a digital processor, an analog processor, a digital circuit designed to process information, an analog circuit designed to process information, a state machine, and/or other mechanisms for electronically processing information. Although processor 16 is shown in FIG. 9 as a single entity, this is for illustrative purposes only. In some implementations, processor 16 may include a plurality of processing units. These processing units may be physically located within the same device, or processor 16 may represent processing functionality of a plurality of devices operating in coordination. For example, in embodiments in which system 10 includes a client/server architecture, processor 16 may include functionality provided by one or more processors of the server computing platform and one or more processors of the client computing platform.

As is shown FIG. 9, processor 16 may be configured to execute one or more computer program modules. The one or more computer program modules may include one or more of a cancerous sample input module 18, a rearrangement correlation module 20, an output module 22, a diagnostic input module 24, a diagnosis module 26, and/or other modules. Processor 16 may be configured to execute modules 18, 20, 22, 24, and/or 26 by software; hardware; firmware; some combination of software, hardware, and/or firmware; and/or other mechanisms for configuring processing capabilities on processor 16.

It should be appreciated that although modules 18, 20, 22, 24, and 26 are illustrated in FIG. 9 as being co-located within a single processing unit, in implementations in which processor 16 includes multiple processing units, one or more of modules 18, 20, 22, 24, and/or 26 may be located remotely from the other modules. The description of the functionality provided by the different modules 18, 20, 22, 24, and/or 26 described below is for illustrative purposes, and is not intended to be limiting, as any of modules 18, 20, 22, 24, and/or 26 may provide more or less functionality than is described. For example, one or more of modules 18, 20, 22, 24, and/or 26 may be eliminated, and some or all of its functionality may be provided by other ones of modules 18, 20, 22, 24, and/or 26. As another example, processor 16 may be configured to execute one or more additional modules that may perform some or all of the functionality attributed below to one of modules 18, 20, 22, 24, and/or 26.

The tumor or cancer sample input module 18 may be configured to receive information related to tumor or cancer samples. The information may include one or more of a sample identification, a tumor or cancer type, a tumor or cancer stage, subject information (e.g., age, sex, race/ethnicity, geographic location, and/or other information), indication of the presence or absence of one or more chromosomal sequence rearrangements, expression amounts of gene coding sequences, and/or other information. The tumor or cancer sample input module 18 may be configured to receive such information via user interface 14, from electronic storage 12, and/or from other sources. For example, tumor or cancer sample input module 18 may be executed on a processor of a server computing platform, and the information may be input to system 10 through one or more client computing platforms associated with system 10. The tumor or cancer sample input module 18 may be configured to store the received information to electronic storage 12. The information may be stored in the form of a spreadsheet, a database, and/or other organizational constructs. The information related to individual samples may be stored in separate records including the information related to corresponding individual ones of the samples.

The rearrangement correlation module 20 may be configured to process the information received by cancerous sample input module 18 to identify correlations between certain somatic chromosomal sequence rearrangements (and/or certain sets of somatic chromosomal sequence rearrangements) and the presence of tumor or cancer. This may include processing the records associated with the individual samples to identify common sets of one or more somatic chromosomal sequence rearrangements that tend to be present in the cancerous samples. In some implementations, the correlation may correlate a common set of one or more somatic chromosomal sequence rearrangements with one or more specific types of tumor or cancer, tumor or cancer stage, progression or worsening (e.g., metastasis), expression amounts of gene coding sequences, and/or other correlations. Some of these chromosomal sequence rearrangements may include one or more of the specific chromosomal sequence rearrangements discussed herein. The rearrangement correlation module 20 may be configured to store the identified correlations to electronic storage 12. The correlations may be stored in the form of a spreadsheet, a database, and/or other organizational constructs.

The output module 22 may be configured to output information related to the processing performed by rearrangement correlation module 20. This may include conveying the correlations identified by rearrangement correlation module 20, and/or conveying other information produced by rearrangement correlation module 20. The output module 22 may convey output the information to users via processor 16. In some implementations in which system 10 includes a client/server architecture. The output module 22 may output the information to users via the client computing platform(s).

The diagnostic input module 24 may be configured to receive information related to samples that may or may not include tumor or cancer. The information may include one or more of a sample identification, care provider information, subject information (e.g., age, sex, race/ethnicity, geographic location, and/or other information), indication of the presence or absence of one or more chromosomal sequence rearrangements, expression amounts of gene coding sequences, and/or other information. The diagnostic input module 24 may be configured to receive such information via user interface 14, from electronic storage 12, and/or from other sources. For example, diagnostic input module 24 may be executed on a processor of a server computing platform, and the information may be input to system 10 through one or more client computing platforms associated with system 10. The diagnostic input module 24 may be configured to store the received information to electronic storage 12. The information may be stored in the form of a spreadsheet, a database, and/or other organizational constructs. The information related to individual samples may be stored in separate records including the information related to corresponding individual ones of the samples.

The diagnosis module 26 may be configured to diagnose the presence of tumor or cancer (or the increased risk of tumor or cancer) in individual samples based on the information received by diagnostic input module 24 and previously identified correlations between tumor or cancer and sets of one or more somatic chromosomal sequence rearrangements. This may include cross-referencing any somatic chromosomal sequence rearrangements present in a sample with one or more sets of somatic chromosomal sequence rearrangements that have previously been correlated with the presence of tumor or cancer (or the increased risk of tumor or cancer). If the somatic chromosomal sequence rearrangement(s) present in a given sample match somatic chromosomal sequence rearrangements that have previously been correlated with tumor or cancer (or the increased risk thereof), the given sample may be identified as having tumor or cancer (or the increased risk thereof). Further diagnostics (e.g., identification of stage, identification of tumor or cancer type, and/or other diagnostics) may be performed based on the previous correlations between the somatic chromosomal sequence rearrangements and tumor or cancer, as described herein. In some implementations, the previously identified correlations between tumor or cancer and sets of one or more somatic chromosomal sequence rearrangements may include the correlations identified by rearrangement correlation module 20.

The output module 22 may be configured to output the diagnosis made by diagnosis module 26. This may include presenting to a user the diagnosis made by diagnosis module 26 based on previously identified correlations tumor or cancer and sets of one or more somatic chromosomal sequence rearrangements.

The risk of, the presence of, or prognosis of a tumor or cancer of a given subject can be used to understand the nature of the tumor or cancer, and to anticipate whether, and to what extent the tumor or cancer will progress or worsen (e.g., metastasize), or respond to treatment. Depending on such information, the subject may be a treated more or less aggressively based upon the anticipated risk, or it may be determined that the recipient can be treated according to less aggressive protocol. Accordingly, the invention provides methods in which risk of tumor or cancer progression or worsening (e.g., metastasize), or response to a given treatment can be anticipated, and such recipients can be treated in accordance with the risk and anticipated treatment response.

The invention provides kits, which kits include, for example, analytes, nucleic acid sequences, primers, probes, antibodies and arrays packaged into a suitable packaging material. Kit components can be used to detect, measure or analyze somatic chromosomal sequence rearrangements, non-rearrangements, or expression of gene coding sequence (e.g., in Tables 1 or 2), for example, a probe, primer pair or antibody that specifically binds to or is capable of detecting, measuring or analyzing a somatic chromosomal sequence rearrangement, non-rearrangement, or expression of a gene coding sequence. Accordingly, in one embodiment, a kit includes an analyte, nucleic acid sequence, primer, probe, antibody or an array that allows detection, measurement or analysis of somatic chromosomal sequence rearrangements (e.g., in Table 1), non-rearrangements, or expression of gene coding sequence (e.g., in Table 2).

The term “packaging material” refers to a physical structure housing one or more components of the kit. The packaging material can maintain the components sterilely, and can be made of material commonly used for such purposes (e.g., paper, corrugated fiber, glass, plastic, foil, ampules, vials, tubes, etc.). A kit can contain a plurality of components, e.g., two or more analytes alone or in combination.

A kit optionally includes a label or insert including a description of the components (type, amounts, etc.), instructions for use in solid phase, in solution, in vitro, in situ, or in vivo, and any other components therein. Labels or inserts can include instructions for practicing any of the methods or other techniques described herein. For example, instructions for detecting, measuring and/or analyzing somatic chromosomal sequence rearrangements (e.g., in Table 1), non-rearrangements, or expression of gene coding sequence (e.g., in Table 2) from a subject's sample. The instructions can additionally indicate that a somatic chromosomal sequence rearrangement, non-rearrangement, or expression of gene coding sequence indicates a higher or lower risk of a tumor or cancer, the type of tumor or cancer, stage or prognosis, and possible treatment regimes appropriate for the tumor or cancer in the subject.

Labels or inserts can include information identifying manufacturer, lot numbers, manufacturer location and date, expiration dates. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Invention kits can additionally include a buffering agent, or a preservative or a stabilizing agent in a formulation containing an analyte (e.g., a nucleic acid sequence, primer, probe or antibody that allows detection, measurement or analysis of expression of a somatic chromosomal sequence rearrangement, non-rearrangement, or expression of gene coding sequence). Each component of the kit can be enclosed within an individual container and all of the various containers can be within a single package.

Kits of the invention can include nucleic acid(s) (e.g., oligonucleotides, primers, or probes) with 100% identity or 100% complementary to all or a portion of a genomic sequence in Table 1 or gene of Table 2, as well as nucleic acid(s) (e.g., oligonucleotides, primers, or probes) having less than 100% identity or less than 100% identity or complementary to all or a portion of a genomic or gene sequence in Tables 1 or 2 (e.g., 60%, 70%, 80%, 85%, 90%, or 95%). Kits therefore include sense and/or anti-sense nucleic acid sequences that hybridize to all or a portion of genomic sequences set forth in Table 1, gene sequences in Table 2.

In one embodiment, a kit includes two or more primer pairs (e.g., 3, 4, 5, 6, 7, 8, 9, 10, 11, 12, 13, 14, 15, 16, 17, 18, 19, 20, etc., or more), each primer pair oppositely oriented to each other, and the primer pairs hybridize to a genomic sequence that includes a potential somatic chromosomal sequence rearrangement. Such primers can be suitable for sequencing and/or amplifying a somatic chromosomal sequence rearrangement. In particular aspects, a somatic chromosomal sequence rearrangement is listed in Table 1.

Kits of the invention can include alternative analytes. In one embodiment, a kit includes a probe that hybridizes to a nucleic acid sequence comprising a somatic chromosomal sequence rearrangement. Such probes can be used to specifically detect, measure or analyze somatic chromosomal sequence rearrangements, including those in Table 1. In particular aspects, a plurality of probes (e.g., 2, 3, 4, 5, 6, 7, 8, 9, 10, 11, 12, 13, 14, 15, 16, 17, 18, 19, 20, etc., or more) that each hybridize to a nucleic acid sequence comprising a somatic chromosomal sequence rearrangement set forth in Table 1 are included in a kit.

Kits of the invention that include analytes need not have all or a portion of the analytes attached or affixed to a support or substrate. In one embodiment, a kit that includes primer pairs or probes, the primer pairs and/or probes are not attached or affixed to a support or substrate.

Kits of the invention can further include other reagents useful in assessing levels of expression of a nucleic acid (e.g., buffers and other reagents for performing PCR reactions, or for detecting binding of a probe to a nucleic acid sequence comprising a somatic chromosomal sequence rearrangement). For example, a kit can also include additional useful materials and substances, such as a standard (e.g., a sample containing a known quantity of a normal (non-rearranged) nucleic acid to which the results can be compared). Kits can additionally include a computer readable media (comprising, for example, a data analysis program, a reference somatic chromosomal sequence rearrangement, or normal non-rearranged sequence, etc.), control samples, and other reagents for obtaining and/or processing sample and analysis, and analyzing genomic nucleic acid for the presence or absence of a somatic chromosomal sequence rearrangement.

The invention provides arrays, which arrays include, for example, one or more analytes, nucleic acid sequences, polynucleotides, oligonucleotides, primers, probes or antibodies affixed to or contained in a support or substrate (e.g., such as a multi-well format, or a multi-well plate or dish). An “array” or “microarray,” which can also be referred to as a “bio-chip,” refers to an arrangement of binding (e.g., hybridizable) analytes, such as polynucleotides, oligonucleotides, primers, probes or antibodies, on a substrate. Such arrays are suitable for quantifying variations in gene expression levels, and are therefore useful for the methods described herein, for example, detecting, measuring or analyzing expression of gene coding sequences (e.g., Table 2).

Typically, in an array an analyte (e.g., nucleic acid sequence, oligonucleotide, probe, primer or antibody) that is a portion of a known gene sequence (single strand, sense or anti-sense), such as a sequence comprising a somatic chromosomal sequence rearrangement, occupies a defined or known address or location on a substrate or support. Accordingly, analytes, such as nucleic acid sequences, polynucleotides, oligonucleotides, primers, probes or antibodies, that bind to a nucleic acid sequence comprising a somatic chromosomal sequence rearrangement, non-rearranged sequences or gene coding sequences (e.g., expression products), can have a defined or known location, position or address on the support or substrate.

Analytes are typically arranged within two or more dimensions of the array. An array can assume different shapes. For example, the array can be regular (such as arranged in uniform rows and columns) or irregular. Thus, in ordered arrays the position/location of each sample is assigned to the sample at the time when it is applied to the array, and a key can correlate each position/location with the appropriate target. An ordered array can be arranged in a symmetrical grid pattern, but samples could be arranged in other patterns (such as in radially distributed lines, spiral lines, or ordered clusters). Arrays usually are computer readable, in that a computer can be programmed to correlate a particular address on the array with sample identity at that position (such as hybridization or binding data, including for instance signal intensity).

An array “format” includes any format in which an analyte can be affixed to or contained in the support or substrate, such as microtiter or multi-well plates or dishes, test tubes, inorganic sheets, dipsticks, etc. The particular format is unimportant. All that is necessary is that an analyte can be affixed to or contained in the support or substrate without affecting the functional behavior of the analyte absorbed thereon.

The support or substrate can be an inert material such as glass or plastic. One such material is an organic polymer such as polypropylene, which is chemically inert and hydrophobic, and has good chemical resistance to a variety of organic acids, organic agents, bases, salts, oxidizing agents, and mineral acids. Additional non-limiting examples include polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulformes, hydroxylated biaxially oriented polypropylene, aminated biaxially oriented polypropylene, thiolated biaxially oriented polypropylene, etyleneacrylic acid, thylene methacrylic acid, and blends or copolymers thereof (e.g., blends of polypropylene, polyethylene, polybutylene, polyisobutylene, etc.).

In one embodiment, an array includes two or more primer pairs, wherein each primer pair is oppositely oriented to each other, and each of the primer pairs hybridize to all or a portion of a nucleic acid sequence that includes a somatic chromosomal sequence rearrangement, such as in Table 1, and wherein each primer pair is affixed to or contained in a support or substrate. In particular aspects, one or more primers of a primer pair have 100% identity or 100% complementary to all or a portion of a genomic sequence in Table 1, or a gene coding sequence in Table 2, or have less than 100% identity or less than 100% complementary to all or a portion of a genomic sequence in Table 1 or a gene coding sequence in Table 2 (e.g., 60%, 70%, 80%, 85%, 90%, or 95% identity or complementary to all or a portion of a genomic or gene coding sequence in Tables 1 or 2. In further particular aspects, the array further includes a probe (or a plurality of probes) that hybridizes to a nucleic acid sequence amplified by one of the primer pairs.

In another embodiment, an array includes two or more probes, wherein each probe hybridizes to all or a portion of a genomic or gene coding sequence in Tables 1 or 2, and wherein each probe is affixed to or contained in a support or substrate. In particular aspects, one or more probes have 100% identity or is 100% complementary to all or a portion of a genomic or gene coding sequence in Tables 1 or 2, or has less than 100% identity or is less than 100% complementary to all or a portion of a genomic or a gene coding sequence in Tables 1 or 2 (e.g., 60%, 70%, 80%, 85%, 90%, or 95% identity or complementary to all or a portion).

Nucleic acid and other analyte arrays can be fabricated either by de novo synthesis on a substrate or by spotting or transporting nucleic acid sequences onto specific locations of substrate. For example, nucleic acid purified and/or isolated from a biological material, such as a sample that includes genomic nucleic acid is hybridized with an array of such oligonucleotides or probes, and then the presence or absence, or amount of target nucleic acid that hybridizes to each oligonucleotide or probe in the array, can be determined.

In further embodiments, an array includes primers and/or probes that hybridize to a plurality of somatic chromosomal sequence rearrangements or gene coding sequences set forth in Tables 1 and/or 2. In further embodiments, an array includes primers and/or probes all of which hybridize to all or a portion of a genomic or gene coding sequence in Tables 1 or 2. In still further embodiments, an array includes a total number of primer pairs and/or probes less than 30,000, less than 20,000, less than 15,000, less than 10,000, less than 5,000, less than 2,500, less than 2,000, less than 1,500, less than 1,000, less than 500, less than 400, less than 300, less than 200, less than 100, less than 50, or less than 25 primer pairs and/or probes.

By way of illustration only, an array of nucleic acids, polynucleotides, oligonucleotides, primers or probes, immobilized on the microchip or microbead, are suitable for hybridization to a nucleic acid sample. Fluorescently labeled cDNA probes (e.g., generated through incorporation of fluorescent nucleotides) are contacted or applied to the array, and allowed to hybridize with specificity to each spot of nucleic acid on the array. After washing to remove non-specifically bound cDNA probes, the array is scanned by a detection method (e.g., by confocal laser microscopy or a CCD camera). Quantitation of hybridization of each array element allows for assessment of the presence or absence of a somatic chromosomal sequence rearrangement.

Arrays can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known (a summary of suitable methods can be found in Matson et al., Anal. Biochem. 217:306-10 (1994)). In still another example, oligonucleotides are synthesized onto the support using conventional chemical techniques for preparing oligonucleotides on solid supports (WO 85/01051, WO 89/10977, and U.S. Pat. No. 5,554,501).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All embodiments, aspects and features disclosed herein may be combined in any combination. Accordingly, all embodiments, aspects and features of the invention, including those described under different embodiments or aspects of the invention, are contemplated to be combined with other embodiments, aspects and features whenever applicable. Each feature disclosed in the specification may be replaced by an alternative feature serving a same, equivalent, or similar purpose. Accordingly, all features of the invention can be substituted or replaced with other equivalent features even if such features are no expressly disclosed herein.

As used herein, the singular forms “a”, “and,” and “the” include plural referents unless the context clearly indicates otherwise. Thus, for example, reference to “a first, second, third, fourth, fifth, etc., genomic sequence rearrangement or analyte” includes a plurality of such first, second, third, fourth, fifth, etc., genomic sequence rearrangements or analytes.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., reference to a range of 1,000-10,000 includes 1,001, 1,002, 1,003, 1,004 . . . 9,996, 9,997, 9,999, 9,998, 9,999, and so forth.

Reference to a series of ranges, for example, reference to a range of 10-20, 20-30, 30-50, 50-100, 100-150, 150-200, 200-250, 250-300, 300-400, 400-500, 500-1000, 1000-2000, 2,000-5,000, 5,000-10,000, 10,000-25,000, 25,000-50,000, 50,000-100,000, 100,000-250,000, 250,000-500,000, 500,000-1,000,000, 1,000,000-5,000,000, 5,000,000-10,000,000, 10,000,000-25,000,000, 25,000,000-50,000,000, 50,000,000-100,000,000 include combinations of combined ranges, such as 10-5,000, 1,000-500,000, 25,000-10,000,000, etc. A series of ranges include both lower and upper ends of those ranges combined into ranges. Thus, for example, reference to a series of ranges such as 10-20, 20-30, 30-50, 50-100, 100-150, 150-200, 200-250, 250-300, 300-400, 400-500, 500-1000, 1000-2000, 2,000-5,000, 5,000-10,000, 10,000-25,000, 25,000-50,000, 50,000-100,000, 100,000-250,000, 250,000-500,000, 500,000-1,000,000, 1,000,000-5,000,000, 5,000,000-10,000,000, 10,000,000-25,000,000, 25,000,000-50,000,000, 50,000,000-100,000,000 includes a range of 10-500, 500-5,000, 500,000-50,000,000, etc.

Reference to a number with more (greater) or less than includes any number greater or less than the reference number, respectively. Thus, for example, a reference to less than 30,000, includes 29,999, 29,998, 29,997, etc. all the way down to the number one (1); and less than 20,000, includes 19,999, 19,998, 19,997, etc. all the way down to the number one (1).

The invention is generally disclosed herein using affirmative language to describe the numerous embodiments. The invention also includes embodiments in which subject matter is excluded, in full or in part, such as substances or materials, method steps and conditions, protocols, or procedures. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### Example 1

This example includes a list of exemplary Somatic Chromosomal Sequence Rearrangements.

TABLE 1
Exemplary Somatic Chromosomal Sequence Rearrangements
Relevant to Cancer Prediction, Diagnosis and Monitoring

| sg_chr | sg_start | sg_end | sg_length | sg_name | ph_chr | ph_start | ph_end | ph_length |
|---|---|---|---|---|---|---|---|---|
| chr1 | 79,177,716 | 84,414,777 | 5,237,062 | sgmt164.10 | chr10 | 24,328,653 | 25,616,569 | 1,287,917 |
| chr1 | 79,177,716 | 84,414,777 | 5,237,062 | sgmt164.10 | chr10 | 26,780,251 | 27,150,556 | 370,306 |
| chr1 | 79,177,716 | 84,414,777 | 5,237,062 | sgmt164.10 | chr1 | 56,498,495 | 59,005,059 | 2,506,565 |
| chr1 | 56,498,495 | 59,005,059 | 2,506,565 | sgmt50.16 | chr3 | 150,104,752 | 150,651,284 | 546,533 |
| chr1 | 56,498,495 | 59,005,059 | 2,506,565 | sgmt50.16 | chr4 | 123,278,910 | 125,141,341 | 1,862,432 |
| chr1 | 56,498,495 | 59,005,059 | 2,506,565 | sgmt50.16 | chr10 | 21,581,611 | 22,244,164 | 662,554 |
| chr1 | 56,498,495 | 59,005,059 | 2,506,565 | sgmt50.16 | chr11 | 18,339,189 | 18,766,440 | 427,252 |
| chr2 | 5,174,608 | 9,099,558 | 3,924,951 | sgmt954.5 | chr6 | 12,953,556 | 13,492,116 | 538,561 |
| chr2 | 5,174,608 | 9,099,558 | 3,924,951 | sgmt954.5 | chr14 | 74,999,855 | 77,279,911 | 2,280,057 |
| chr2 | 57,825,183 | 61,899,453 | 4,074,271 | sgmt963.5 | chr1 | 182,351,950 | 182,647,216 | 295,267 |
| chr3 | 72,517,657 | 74,474,129 | 1,956,473 | sgmt1257.36 | chr16 | 4,902,761 | 5,140,847 | 238,087 |
| chr5 | 156,565,132 | 158,632,403 | 2,067,272 | sgmt1511.17 | chr6 | 12,953,556 | 13,492,116 | 538,561 |
| chr6 | 7,047,303 | 9,164,260 | 2,116,958 | sgmt1596.6 | chr5 | 127,469,416 | 128,152,120 | 682,705 |
| chr7 | 155,264,117 | 157,210,205 | 1,946,089 | sgmt1687.16 | chr2 | 204,546,848 | 205,747,855 | 1,201,008 |
| chr8 | 92,587,940 | 94,938,420 | 2,350,481 | sgmt1782.22 | chr8 | 95,158,106 | 97,246,188 | 2,088,083 |
| chr8 | 92,587,940 | 94,938,420 | 2,350,481 | sgmt1782.22 | chr8 | 100,204,991 | 101,300,870 | 1,095,880 |
| chr8 | 92,587,940 | 94,938,420 | 2,350,481 | sgmt1782.22 | chr8 | 73,524,706 | 74,020,731 | 496,026 |
| chr11 | 30,351,542 | 32,975,808 | 2,624,267 | sgmt305.3 | chr11 | 38,573,713 | 38,786,646 | 212,934 |
| chr12 | 41,040,453 | 45,974,198 | 4,933,746 | sgmt385.5 | chr12 | 21,680,651 | 25,047,423 | 3,366,773 |
| chr13 | 53,236,066 | 55,250,543 | 2,014,478 | sgmt493.25 | chr13 | 61,279,887 | 61,544,511 | 264,525 |
| chr13 | 58,902,901 | 61,141,887 | 2,238,987 | sgmt493.29 | chr5 | 131,975,089 | 132,437,799 | 462,711 |
| chr15 | 94,878,945 | 99,073,175 | 4,194,231 | sgmt576.21 | chr6 | 97,236,933 | 100,229,929 | 2,992,997 |
| chr16 | 6,703,581 | 9,024,395 | 2,320,815 | sgmt677.6 | chr16 | 6,186,373 | 6,467,032 | 280,660 |
| chr18 | 18,877,624 | 23,308,408 | 4,430,785 | sgmt788.7 | chr20 | 30,073,091 | 31,440,748 | 1,367,658 |
| chr18 | 18,877,624 | 23,308,408 | 4,430,785 | sgmt788.7 | chr18 | 31,179,004 | 31,808,361 | 629,358 |
| chr18 | 18,877,624 | 23,308,408 | 4,430,785 | sgmt788.7 | chr18 | 68,968,542 | 69,294,308 | 325,767 |
| chr19 | 30,115,800 | 33,770,238 | 3,654,439 | sgmt842.2 | chr19 | 29,570,255 | 30,082,475 | 512,221 |

| sg_chr | ph_name | cell | bk_chr1 | bk_pos1 | bk_chr2 | bk_pos2 | Tissue | Raf |
|---|---|---|---|---|---|---|---|---|
| chr1 | sgmt174.12 | PD3646a | chr1 | 81,401,662 | chr10 | 25,256,020 | Pancreas | Cambell |
| | | | | | | | | 2010 |
| chr1 | sgmt174.16 | PD3646a | chr1 | 81,394,007 | chr1 | 26,947,481 | Pancreas | Cambell |
| | | | | | | | | 2010 |
| chr1 | sgmt50.16 | PD3646a | chr1 | 58,953,188 | chr1 | 82,127,414 | Pancreas | Cambell |
| | | | | | | | | 2010 |
| chr1 | sgmt1308.2 | PD3646a | chr1 | 57,028,848 | chr3 | 150,598,333 | Pancreas | Cambell |
| | | | | | | | | 2010 |
| chr1 | sgmt1431.27 | NCIH209 | chr1 | 57,856,241 | chr4 | 124,307,333 | Lung | Pleasance |
| | | | | | | | | 2010 |
| chr1 | sgmt174.9 | PD3646a | chr1 | 56,498,532 | chr1 | 22,016,121 | Pancreas | Cambell |
| | | | | | | | | 2010 |
| chr1 | sgmt255.20 | NCIH209 | chr1 | 57,907,475 | chr1 | 18,707,698 | Lung | Pleasance |
| | | | | | | | | 2010 |
| chr2 | sgmt1596.12 | Co108C | chr2 | 7,351,259 | chr6 | 13,191,406 | Colon | Leary |
| | | | | | | | | 2010 |
| chr2 | sgmt543.35 | PD3693a | chr2 | 6,911,296 | chr14 | 77,007,664 | Breast | Stephens |
| | | | | | | | | 2009 |
| chr2 | sgmt47.20 | PD3664a | chr2 | 59,879,542 | chr1 | 182,629,775 | Breast | Stephens |
| | | | | | | | | 2009 |
| chr3 | sgmt677.4 | PD3668a | chr3 | 73,721,833 | chr16 | 5,039,914 | Breast | Stephens |
| | | | | | | | | 2009 |
| chr5 | sgmt1596.12 | Co108C | chr5 | 157,590,959 | chr6 | 13,191,312 | Colon | Leary |
| | | | | | | | | 2010 |
| chr6 | sgmt1585.2 | PD3690a | chr6 | 7,677,860 | chr5 | 128,055,995 | Breast | Stephens |
| | | | | | | | | 2009 |
| chr7 | sgmt996.44 | PD3687a | chr7 | 156,711,815 | chr2 | 205,703,824 | Breast | Stephens |
| | | | | | | | | 2009 |
| chr8 | sgmt1782.23 | PD3641a | chr8 | 93,016,796 | chr8 | 96,781,510 | Pancreas | Cambell |
| | | | | | | | | 2010 |
| chr8 | sgmt1783.8 | PD3828c | chr8 | 93,285,960 | chr8 | 100,257,044 | Pancreas | Cambell |
| | | | | | | | | 2016 |
| chr8 | sgmt1828.14 | PD3644a | chr8 | 94,770,703 | chr8 | 73,697,575 | Pancreas | Cambell |
| | | | | | | | | 2010 |
| chr11 | sgmt256.8 | PD3642a | chr11 | 30,559,116 | chr11 | 38,723,130 | Pancreas | Cambell |
| | | | | | | | | 2010 |
| chr12 | sgmt348.25 | PD3642a | chr12 | 41,211,851 | chr12 | 23,338,606 | Pancreas | Cambell |
| | | | | | | | | 2010 |
| chr13 | sgmt493.30 | B7C | chr13 | 53,261,978 | chr13 | 61,540,524 | Breast | Leary |
| | | | | | | | | 2010 |
| chr13 | sgmt1511.4 | PD3667a | chr13 | 59,467,938 | chr5 | 132,402,848 | Breast | Stephens |
| | | | | | | | | 2009 |
| chr15 | sgmt1660.11 | PD3664a | chr6 | 99,914,848 | chr15 | 98,421,254 | Breast | Stephens |
| | | | | | | | | 2009 |
| chr16 | spt677.5 | Hx403x | chr16 | 6,787,735 | chr16 | 6,403,640 | Breast | Leary |
| | | | | | | | | 2010 |
| chr18 | sgmt1113.3 | B5C | chr18 | 20,887,987 | chr20 | 30,128,283 | Breast | Leary |
| | | | | | | | | 2010 |
| chr18 | sgmt788.17 | PD3645a | chr18 | 21,025,585 | chr18 | 31,657,606 | Pancreas | Cambell |
| | | | | | | | | 2010 |
| chr18 | sgmt788.28 | PD3640a | chr18 | 20,071,457 | chr18 | 69,134,293 | Pancreas | Cambell |
| | | | | | | | | 2010 |
| chr19 | sgmt842.1 | PD3827d | chr19 | 30,081,434 | chr19 | 30,884,168 | Pancreas | Cambell |
| | | | | | | | | 2012 |

Legend
sg_* coordinates, length, name of the syntenic segment containing the regulating DNA
ph_* coordinates, length, name of the Philadelphia segment containing the dysregulated gene
cell name of the tumor cell
bk_* coordinates of the breakpoint (2 ends)
Tissue Lung, breast, colon, pancreas
Ref Reference of the cell data
sgmt788.7 Indicates that the segment is broken by different breakpoits, in the same cell or in different cells
sgmt1596.12 Indicates that the Philadelphia segment is broken by different breakpoits, in the same cell or in different cells

### Example 2

This example includes a list of exemplary gene coding sequences relevant to the invention.

TABLE 2
Exemplary Genes Relevant to Cancer Prediction,
Diagnosis and Monitoring

| Symbol | GENE Name |
|---|---|
| ADAM19 | ADAM metallopeptidase domain 19 preproprotein |
| ASXL1 | additional sex combs like 1 isoform 1 |
| BCAT1 | branched chain aminotransferase 1, cytosolic |
| BCL11A | B-cell CLL/lymphoma 11A |
| BMP6 | bone morphogenetic protein 6 preproprotein |
| CABLES1 | Cdk5 and Abl enzyme substrate 1 isoform 1 |
| CCNE1 | Homo sapiens cDNA FLJ75709 complete cds, highly |
| | similar to Homo sapiens cyclin |
| CCNE2 | cyclin E2 |
| CD28 | Homo sapiens T-cell specific surface glycoprotein |
| | CD28 isoform 1 (CD28) gene, co |
| CLRN1 | clarin |
| CMAS | cytidine monophospho-N-acetylneuraminic acid |
| CNTN1 | contactin 1 isoform 1 precursor |
| COX6C | cytochrome c oxidase subunit VIc proprotein |
| DAB1 | disabled homolog 1 |
| DNMT3B | DNA cytosine-5 methyltransferase 3 beta isoform |
| ESRRB | estrogen-related receptor beta |
| FGF2 | fibroblast growth factor 2 |
| FLVCR2 | feline leukemia virus subgroup C cellular |
| FOS | v-fos FBI murine osteosarcoma viral oncogene |
| GDF6 | growth differentiation factor 6 precursor |
| GLUL | glutamine synthetase |
| ICOS | inducible T-cell co-stimulator precursor |
| ID1 | inhibitor of DNA binding 1 |
| IL2 | interleukin 2 precursor |
| ITK | IL2-inducible T-cell kinase |
| KIAA1109 | Homo sapiens mRNA for KIAA1109 protein, partial cds. |
| LAMA3 | laminin alpha 3 subunit isoform 4 |
| LECT1 | leukocyte cell derived chemotaxin 1 isoform 1 |
| LMBR1 | limb region 1 protein |
| MAPRE1 | microtubule-associated protein, RP/EB family, |
| MLH3 | mutL homolog 3 isoform 1 |
| MLLT10 | myeloid/lymphoid or mixed-lineage leukemia |
| MPPED2 | metallophosphoesterase domain containing 2 |
| NELL2 | NEL-like protein 2 isoform a |
| NUDT6 | nudix-type motif 6 isoform a |
| PAX6 | paired box gene 6 isoform b |
| PGF | placental growth factor, vascular endothelial |
| PLAGL2 | pleiomorphic adenoma gene-like 2 |
| PPL | periplakin |
| RAD50 | Homo sapiens RAD50-2 protein (RAD50) mRNA, |
| | alternatively spliced, complete cds |
| RAD54B | RAD54 homolog B |
| RBBP8 | retinoblastoma binding protein 8 isoform b |
| RCN1 | reticulocalbin 1 precursor |
| RNASEL | ribonuclease L |
| RNF144A | ring finger protein 144 |
| RUNX1T1 | acute myelogenous leukemia 1 translocation 1 |
| SHH | sonic hedgehog preproprotein |
| SHROOM1 | shroom family member 1 |
| SOX11 | SRY-box 11 |
| SOX30 | SRY (sex determining region Y)-box 30 |
| SOX5 | SRY (sex determining region Y)-box 5 |
| TBC1D7 | TBC1 domain family, member 7 isoform b |
| TGFB3 | transforming growth factor, beta 3 precursor |
| TSG101 | tumor susceptibility gene 101 |
| VPS13B | vacuolar protein sorting 13B |
| VRK2 | vaccinia related kinase 2 |
| WT1 | Wilms tumor 1 |

1. A method for predicting the presence or absence of a tumor or cancer in a subject or determining the risk of a tumor or cancer in a subject, comprising:
a) analyzing genomic nucleic acid for the presence or absence of a somatic chromosomal sequence rearrangement predictive of the presence of tumor or cancer or an increased risk of tumor or cancer; wherein the somatic chromosomal sequence rearrangement is in a genomic synteny block sequence, and wherein all or a portion of the genomic synteny block sequence is structurally rearranged to be in an altered proximity to a gene coding sequence;
b) wherein the presence of the somatic chromosomal sequence rearrangement is predictive of the presence of tumor or cancer in the subject or an increased risk of tumor or cancer in the subject; and
c) wherein the absence of the somatic chromosomal sequence rearrangement is predictive of the absence of tumor or cancer in the subject or a reduced risk of tumor or cancer in the subject,
thereby predicting the presence or absence of tumor or cancer or determining the risk of tumor or cancer in the subject.

2. The method of claim 1, wherein the sequence rearrangement is in any of: chromosome 1, in a sequence region from about 79,177,716 to about 84,414,777; chromosome 1, in a sequence region from about 56,498,495 to about 59,005,059; chromosome 2, in a sequence region from about 5,174,608 to about 9,099,558; chromosome 2, in a sequence region from about 57,825,183 to about 61,899,453; chromosome 3, in a sequence region from about 72,517,657 to about 74,474,129; chromosome 5, in a sequence region from about 156,565,132 to about 158,632,403; chromosome 6, in a sequence region from about 7,047,303 to about 9,164,260; chromosome 7, in a sequence region from about 155,264,117 to about 157,210,205; chromosome 8, in a sequence region from about 92,587,940 to about 94,938,420; chromosome 11, in a sequence region from about 30,351,542 to about 32,975,808; chromosome 12, in a sequence region from about 41,040,453 to about 45,974,198; chromosome 13, in a sequence region from about 53,236,066 to about 55,250,543; chromosome 13, in a sequence region from about 58,902,901 to about 61,141,887; chromosome 15, in a sequence region from about 94,878,945 to about 99,073,175; chromosome 16, in a sequence region from about 6,703,581 to about 9,024,395; chromosome 18, in a sequence region from about 18,877,624 to about 23,308,408; chromosome 19, in a sequence region from about 30,115,800 to about 33,770,238, of all or a part of any of the foregoing genomic synteny block sequences, wherein numerical coordinates for said genomic synteny block sequence are as defined in the Human Genome Reference Consortium, Version GRCh37.

3.-5. (canceled)

6. The method of claims 1, wherein the sequence rearrangement comprises a sequence translocated to: chromosome 1, in a sequence region from about 56,498,495 to about 59,005,059; chromosome 1, in a sequence region from about 182,351,950 to about 182,647,216; chromosome 2, in a sequence region from about 204,546,848 to about 205,747,855; chromosome 3, in a sequence region from about 150,104,752 to about 150,651,284; chromosome 4, in a sequence region from about 123,278,910 to about 125,141,341; chromosome 5, in a sequence region from about 127,469,416 to about 128,152,120; chromosome 5, in a sequence region from about 131,975,089 to about 132,437,799; chromosome 6, in a sequence region from about 12,953,556 to about 13,492,116; chromosome 6, in a sequence region from about 97,236,933 to about 100,229,929; chromosome 8, in a sequence region from about 95,158,106 to about 97,246,188; chromosome 8, in a sequence region from about 100,204,991 to about 101,300,870; chromosome 8, in a sequence region from about 73,524,706 to about 74,020,731; chromosome 10, in a sequence region from about 24,328,653 to about 25,616,569; chromosome 10, in a sequence region from about 26,780,251 to about 27,150,556; chromosome 10, in a sequence region from about 21,581,611 to about 22,244,164; chromosome 11, in a sequence region from about 18,339,189 to about 18,766,440; chromosome 11, in a sequence region from about 38,573,713 to about 38,786,646; chromosome 12, in a sequence region from about 21,680,651 to about 25,047,423; chromosome 13, in a sequence region from about 61,279,987 to about 61,544,511; chromosome 14, in a sequence region from about 74,999,855 to about 77,279,911; chromosome 16, in a sequence region from about 4,902,761 to about 5,140,847; chromosome 16, in a sequence region from about 6,186,373 to about 6,467,032; chromosome 18, in a sequence region from about 31,179,004 to about 31,808,361; chromosome 18, in a sequence region from about 68,968,542 to about 69,294,308; chromosome 19, in a sequence region from about 29,570,255 to about 30,082,475; chromosome 20, in a sequence region from about 30,073,091 to about 31,440,748, wherein numerical coordinates for said genomic synteny block sequences are as defined in the Human Genome Reference Consortium, Version GRCh37.

7. The method of claim 1, wherein the sequence rearrangement comprises a break in a sequence region from about 56,498,495 to about 59,005,059 of chromosome 1, and translocation to chromosome 3, in a sequence region from about 150,104,752 to about 150,651,284; a break in a sequence region from about 56,498,495 to about 59,005,059 of chromosome 1, and translocation to chromosome 4, in a sequence region from about 123,278,910 to about 125,141,341; a break in a sequence region from about 56,498,495 to about 59,005,059 of chromosome 1, and translocation to chromosome 10, in a sequence region from about 21,581,611 to about 22,244,164; a break in a sequence region from about 56,498,495 to about 59,005,059 of chromosome 1, and translocation to chromosome 11, in a sequence region from about 18,339,189 to about 18,766,440; a break in a sequence region from about 79,177,716 to about 84,414,777 of chromosome 1, and translocation to chromosome 1, in a sequence region from about 56,498,495 to about 59,005,059; a break in a sequence region from about 79,177,716 to about 84,414,777 of chromosome 1, and translocation to chromosome 10, in a sequence region from about 24,328,653 to about 25,616,569; a break in a sequence region from about 79,177,716 to about 84,414,777 of chromosome 1, and translocation to chromosome 10, in a sequence region from about 26,780,251 to about 27,150,556; a break in a sequence region from about 5,174,608 to about 9,099,558 of chromosome 2, and translocation to chromosome 6, in a sequence region from about 12,953,556 to about 13,492,116; a break in a sequence region from about 5,174,608 to about 9,099,558 of chromosome 2, and translocation to chromosome 14, in a sequence region from about 74,999,855 to about 77,279,911; a break in a sequence region from about 57,825,183 to about 61,899,453 of chromosome 2, and translocation to chromosome 1, in a sequence region from about 182,351,950 to about 182,647,216; a break in a sequence region from about 72,517,657 to about 74,474,129 of chromosome 3, and translocation to chromosome 16, in a sequence region from about 4,902,761 to about 5,140,847; a break in a sequence region from about 156,565,132 to about 158,632,403 of chromosome 5, and translocation to chromosome 6, in a sequence region from about 12,953,556 to about 13,492,116; a break in a sequence region from about 7,047,303 to about 9,164,260 of chromosome 6, and translocation to chromosome 5, in a sequence region from about 127,469,416 to about 128,152,120; a break in a sequence region from about 155,264,117 to about 157,210,205 of chromosome 7, and translocation to chromosome 2, in a sequence region from about 204,546,848 to about 205,747,855; a break in a sequence region from about 92,587,940 to about 94,938,420 of chromosome 8, and translocation to chromosome 8, in a sequence region from about 95,158,106 to about 97,246,188; a break in a sequence region from about 92,587,940 to about 94,938,420 of chromosome 8, and translocation to chromosome 8, in a sequence region from about 100,204,991 to about 101,300,870; a break in a sequence region from about 92,587,940 to about 94,938,420 of chromosome 8, and translocation to chromosome 8, in a sequence region from about 73,524,706 to about 74,020,731; a break in a sequence region from about 30,351,542 to about 32,975,808 of chromosome 11, and translocation to chromosome 11, in a sequence region from about 38,573,713 to about 38,786,646; a break in a sequence region from about 41,040,453 to about 45,974,198 of chromosome 12, and translocation to chromosome 12, in a sequence region from about 21,680,651 to about 25,047,423; a break in a sequence region from about 53,236,066 to about 55,250,543 of chromosome 13, and translocation to chromosome 13, in a sequence region from about 61,279,987 to about 61,544,511; a break in a sequence region from about 58,902,901 to about 61,141,887 of chromosome 13, and translocation to chromosome 5, in a sequence region from about 131,975,089 to about 132,437,799; a break in a sequence region from about 94,878,945 to about 99,073,175 of chromosome 15, and translocation to chromosome 6, in a sequence region from about 97,236,933 to about 100,229,929; a break in a sequence region from about 6,703,581 to about 9,024,395 of chromosome 16, and translocation to chromosome 16, in a sequence region from about 6,186,373 to about 6,467,032; a break in a sequence region from about 18,877,624 to about 23,308,408 of chromosome 18, and translocation to chromosome 18, in a sequence region from about 31,179,004 to about 31,808,361; a break in a sequence region from about 18,877,624 to about 23,308,408 of chromosome 18, and translocation to chromosome 18, in a sequence region from about 68,968,542 to about 69,294,308; a break in a sequence region from about 18,877,624 to about 23,308,408 of chromosome 18, and translocation to chromosome 20, in a sequence region from about 30,073,091 to about 31,440,748; a break in a sequence region from about 30,115,800 to about 33,770,238 of chromosome 19, and translocation to chromosome 19, in a sequence region from about 29,570,255 to about 30,082,475, wherein numerical coordinates for said genomic sequence regions are as defined in the Human Genome Reference Consortium, Version GRCh37.

8.-13. (canceled)

14. The method of claim 1, wherein the sequence rearrangement comprises an intra-chromosomal or inter-chromosomal rearrangement.

15. The method of claim 1, wherein the sequence rearrangement comprises a sequence translocation, tandem duplication, inverted duplication, or deletion.

16.-18. (canceled)

19. (canceled)

20. The method of claim 1, wherein the genomic synteny block sequence comprises a sequence having of length of 1,000 or more, 2,000 or more, 5,000 or more, 10,000 or more, 25,000 or more, 50,000 or more, 100,000 or more, 200,000 or more, 300,000 or more, 400,000 or more, 500,000 or more, 600,000 or more, 700,000 or more, 800,000 or more, 900,000 or more, or 1,000,000 or more, base pairs.

21.-23. (canceled)

24. The method of claim 1, wherein the genomic synteny block sequence comprises a density of non-coding sequences, segments or elements of at least 3 to 1 gene coding sequences, segments or elements, per 50,000 base pairs.

25.-31. (canceled)

32. The method of claim 1, wherein the gene coding sequence comprises ADAM19, ASXL1, BCAT1, BCL11A, BMP6, CABLES1, CCNE1, CCNE2, CD28, CLRN1, CMAS, CNTN1, COX6C, DAB1, DNMT3B, ESRRB, FGF2, FLVCR2, FOS, GDF6, GLUL, ICOS, ID1, IL2, ITK, KIAA1109, LAMA3, LECT1, LMBR1, MAPRE1, MLH3, MLLT10, MPPED2, NELL2, NUDT6, PAX6, PGF, PLAGL2, PPL, RAD50, RAD54B, RBBP8, RCN1, RNASEL, RNF144A, RUNX1T1, SHH, SHROOM1, SOX11, SOX30, SOX5, TBC1D7, TGFB3, TSG101, VPS13B, VRK2, WIT1, or WT1.

33.-36. (canceled)

37. The method of claim 1, wherein a number equal to or greater than a threshold number of somatic chromosomal sequence rearrangements indicates the presence of or an increased risk of tumor or cancer.

38. The method of claim 1, wherein a number equal to or less than a threshold number of somatic chromosomal sequence rearrangements indicates the absence of or a reduced risk of tumor or cancer.

39. The method of claim 1, wherein the analyzing comprises contact of the genomic nucleic acid or a nucleic acid derived from the genomic nucleic acid, with an analyte that detects the presence or detects the absence of the somatic chromosomal sequence rearrangement.

40. (canceled)

41. The method of claim 1, wherein the analyzing comprises hybridization with an oligo- or poly-nucleotide probe to the somatic chromosomal sequence rearrangement, or a nucleic acid derived from the somatic chromosomal sequence rearrangement.

42. The method of claim 1, wherein the analyzing comprises hybridization with a primer pair that flanks the sequence region of the somatic chromosomal sequence rearrangement, or a nucleic acid derived from the somatic chromosomal sequence rearrangement, and subsequent sequence amplification of a sequence comprising the somatic chromosomal sequence rearrangement or the nucleic acid derived from the somatic chromosomal sequence rearrangement.

43.-47. (canceled)

48. The method of claim 1, further comprising assigning a risk score based upon the presence or absence of one or more somatic chromosomal sequence rearrangements.

49. The method of claim 1, further comprising assigning a risk score based upon the number of somatic chromosomal sequence rearrangements, or the type of somatic chromosomal sequence rearrangements.

50. The method of claim 48, wherein the risk scores are recorded or stored on an electronic or computer readable medium, or in a database or other organizational construct.

51. (canceled)

52. A kit, comprising one or more nucleic acid probes, wherein each probe hybridizes to a nucleic acid comprising a chromosomal sequence rearrangement within one or more genomic synteny block sequences selected from: chromosome 1, in a sequence from about 79,177,716 to about 84,414,777; chromosome 1, in a sequence region from about 56,498,495 to about 59,005,059; chromosome 2, in a sequence region from about 5,174,608 to about 9,099,558; chromosome 2, in a sequence region from about 57,825,183 to about 61,899,453; chromosome 3, in a sequence region from about 72,517,657 to about 74,474,129; chromosome 5, in a sequence region from about 156,565,132 to about 158,632,403; chromosome 6, in a sequence region from about 7,047,303 to about 9,164,260; chromosome 7, in a sequence region from about 155,264,117 to about 157,210,205; chromosome 8, in a sequence region from about 92,587,940 to about 94,938,420; chromosome 11, in a sequence region from about 30,351,542 to about 32,975,808; chromosome 12, in a sequence region from about 41,040,453 to about 45,974,198; chromosome 13, in a sequence region from about 53,236,066 to about 55,250,543; chromosome 13, in a sequence region from about 58,902,901 to about 61,141,887; chromosome 15, in a sequence region from about 94,878,945 to about 99,073,175; chromosome 16, in a sequence region from about 6,703,581 to about 9,024,395; chromosome 18, in a sequence region from about 18,877,624 to about 23,308,408; chromosome 19, in a sequence region from about 30,115,800 to about 33,770,238, and the sequence rearrangement is all or a portion of any of the foregoing genomic synteny block sequences; and wherein at least one of the probes can detect the presence of a chromosomal sequence rearrangement within the foregoing genomic synteny block sequence, wherein numerical coordinates for said genomic synteny block sequences are as defined in the Human Genome Reference Consortium, Version GRCh37.

53.-62. (canceled)

63. A system configured to identify samples having somatic chromosomal sequence rearrangements indicative of a tumor or cancer, the system comprising:
a) electronic storage storing a plurality of somatic chromosomal sequence rearrangements indicative of a tumor or cancer; and
b) one or more processors configured to receive analysis of a sample indicating the presence or absence one or more somatic chromosomal sequence rearrangements in the sample, to compare any somatic chromosomal sequence rearrangements in the sample with the stored plurality of somatic chromosomal sequence rearrangements indicative of a tumor or cancer, and, responsive to a somatic chromosomal sequence rearrangements in the sample matching one of the stored somatic chromosomal sequence rearrangements, to identify the sample as having a tumor or cancer.

64. The system of claim 63, wherein the plurality of somatic chromosomal sequence rearrangements include one or more somatic chromosomal rearrangements within a genomic synteny block sequence selected from: chromosome 1, in a sequence from about 79,177,716 to about 84,414,777; chromosome 1, in a sequence region from about 56,498,495 to about 59,005,059; chromosome 2, in a sequence region from about 5,174,608 to about 9,099,558; chromosome 2, in a sequence region from about 57,825,183 to about 61,899,453; chromosome 3, in a sequence region from about 72,517,657 to about 74,474,129; chromosome 5, in a sequence region from about 156,565,132 to about 158,632,403; chromosome 6, in a sequence region from about 7,047,303 to about 9,164,260; chromosome 7, in a sequence region from about 155,264,117 to about 157,210,205; chromosome 8, in a sequence region from about 92,587,940 to about 94,938,420; chromosome 11, in a sequence region from about 30,351,542 to about 32,975,808; chromosome 12, in a sequence region from about 41,040,453 to about 45,974,198; chromosome 13, in a sequence region from about 53,236,066 to about 55,250,543; chromosome 13, in a sequence region from about 58,902,901 to about 61,141,887; chromosome 15, in a sequence region from about 94,878,945 to about 99,073,175; chromosome 16, in a sequence region from about 6,703,581 to about 9,024,395; chromosome 18, in a sequence region from about 18,877,624 to about 23,308,408; chromosome 19, in a sequence region from about 30,115,800 to about 33,770,238, wherein numerical coordinates for said genomic synteny block sequences are as defined in the Human Genome Reference Consortium, Version GRCh37.

65.-66. (canceled)

67. The system of claim 63, wherein the system includes 2, 3, 4, 5, 6, 7, 8, 9, 10, 11, 12, 13, 14, 15, 16, 17, 18, 19, 20 or more somatic chromosomal sequence rearrangements associated with the tumor or cancer.

68.-71. (canceled)

72. The system of claim 63, wherein a risk score is assigned to the tumor or cancer based upon the presence or absence of one or more somatic chromosomal sequence rearrangements.

73. The system of claim 63, wherein a risk score is assigned to the tumor or cancer based upon the number of somatic chromosomal sequence rearrangements, or the type of somatic chromosomal sequence rearrangements.

74. The system of claim 63, wherein the electronic storage comprises a computer readable medium.

75. The system of claim 63, wherein the said processor further comprises a data entry module or a data query module.

76. The method of claim 1, wherein the method predicts the presence or absence of a tumor or cancer with an accuracy of at least 60%.

77. The method of claim 1, wherein a threshold number of somatic chromosomal sequence rearrangements predicts the presence or absence of a tumor or cancer.

78. The method of claim 39, wherein the analyte comprises a primer pair, an oligo- or poly-nucleotide probe, or an antibody or antigen binding fragment thereof.

79. The method of claim 1, further comprising creating a report of the presence or absence of the tumor or cancer, the type or tumor or cancer, or progression or severity of the tumor or cancer.

80. The method of claim 49, wherein the risk scores are recorded or stored on an electronic or computer readable medium, or in a database or other organizational construct.
